(19) United States
(12) Patent Application Publication
(10) Pub. No.: US 2025/0366989 A1
(43) Pub. Date: Dec. 4, 2025

(54) DELIVERY SYSTEM IMPLANT CINCH AND RELEASE MECHANISM

(71) Applicant: Medtronic, Inc., Minneapolis, MN (US)

(72) Inventors: David A. Grossman, Santa Rosa, CA (US); Hailey Queen, Santa Rosa, CA (US); Erik C. Griswold, Penngrove, CA (US); Stacey M. Plasencia, Rohnert Park, CA (US)

(21) Appl. No.: 18/875,119

(22) PCT Filed: Jun. 12, 2023
(86) PCT No.: PCT/IB2023/056057

§ 371 Date: Dec. 13, 2024

Related U.S. Application Data
(60) Provisional application No. 63/366,427, filed on Jun. 15, 2022.

(51) Int. Cl. A61F 2/24

(52) U.S. Cl. CPC A61F 2/2445; A61F 2/2466; A61F 2220/0075

(57) ABSTRACT

In some examples, a medical system includes a prosthetic device configured to expand radially outward to position a valve assembly to control blood flow through an annulus of a heart valve. The prosthetic device includes a cinch member configured to control a device perimeter defined by the prosthetic device. In some examples, the cinch member defines a closed loop substantially around a valve axis of the prosthetic device. In examples, the prosthetic device includes an outer support configured to expand to engage the annulus of the heart valve. In examples, the outer support defines the perimeter. The medical system may include an engagement device configured to cause the cinch member to control the device perimeter when the prosthetic device is positioned within a chamber of a heart of a patient.

### TECHNICAL FIELD

The disclosure relates to valve prostheses and more particularly to delivery systems for a transcatheter heart valve prosthesis.

### BACKGROUND

Systems, devices, and techniques used to repair heart valves are one example of a category of medical devices to which this disclosure relates, although this disclosure also relates to the delivery of other medical devices. Native heart valves can be affected by several medical conditions. For example, mitral valves can be affected by mitral valve regurgitation, mitral valve prolapse and mitral valve stenosis. One method of treatment includes replacement of the heart valve by implanting a prosthetic heart valve in the heart in place of the native mitral valve. Another method of treatment includes repair, bypassing, or replacement of a previously implanted prosthetic heart valve. In some cases, one or more heart valve prostheses may be implanted percutaneously using delivery systems. In some cases, a heart valve prosthesis may be constrained in an initial delivery configuration to allow for the percutaneous delivery via a catheter, with the prosthetic heart valve assuming a relatively small cross-sectional dimension in an initial delivery configuration for delivery through the vasculature of a patient. In some cases, once delivered and placed in the target site, the heart valve prosthesis may be deployed, expanding to assume a larger cross-sectional dimension.

### SUMMARY

In some examples, the disclosure is directed to a system including a prosthetic device to engage an annulus of a heart valve of a heart. The prosthetic device is configured to cause a valve axis defined by the prosthetic device to pass through the annulus when the prosthetic device engages the annulus. The prosthetic device includes a cinch member including a member body including a first body portion and a second body portion defining a closed loop. The first body portion is configured to translate within one or more passages defined by the prosthetic device. The prosthetic device defines a device perimeter surrounding the valve axis and the first body portion is configured to reduce a perimeter dimension of the device perimeter when an engagement device exerts a force on the second body portion.

In some examples, the disclosure is directed to a system for delivering a heart valve prosthesis. The system includes an engagement device including an engagement device body defining a capture portion and a prosthetic device configured to engage an annulus of a heart valve of a heart. The prosthetic device is configured to cause a valve axis defined by the prosthetic device to pass through the annulus when the prosthetic device engages the annulus. The prosthetic device includes a cinch member including a member body including a first body portion and a second body portion defining a closed loop. The first body portion is configured to translate within one or more passages defined by the prosthetic device. The prosthetic device defines a device perimeter surrounding the valve axis, and the first body portion is configured to reduce a perimeter dimension of the device perimeter when the capture portion engages the second body portion and the engagement device exerts a force on the second body portion. The capture portion is configured to disengage from the second body portion.

In some examples, the disclosure is directed to a method of delivering a prosthetic device. The method includes expanding a perimeter defined by prosthetic device using an engagement device engaged with a cinch member. The prosthetic device configured to engage an annulus of a heart valve of a heart, and the prosthetic device is configured to cause a valve axis defined by the prosthetic device to pass through the annulus when the prosthetic device engages the annulus. The cinch member includes a member body including a first body portion and a second body portion defining a closed loop. The first body portion is disposed within one or more passages defined by the prosthetic device, and the second body portion is configured to be engaged by an engagement device. The prosthetic device defines a device perimeter surrounding the valve axis, and the first body portion is configured to reduce a perimeter dimension defined by the device perimeter when the engagement device exerts a force on the second body portion. The method includes disengaging the prosthetic device from the engagement device.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a conceptual diagram illustrating an example delivery system including a prosthetic device in a delivery configuration.

FIG. 1B is a conceptual diagram of the delivery system of FIG. 1A with the prosthetic device in an expanded configuration.

FIG. 2 is a perspective view illustrating an example prosthetic device including a cinch member.

FIG. 3A is a schematic cross-sectional view illustrating the example prosthetic device of FIG. 2, the cross-section being taken through an axis L of the prosthetic device.

FIG. 3B is a top plan view of the prosthetic device of FIG. 3A.

FIG. 4 is a perspective view illustrating the underlying structure of the prosthetic device of FIG. 2.

FIG. 5 is a schematic side view illustrating an example capture portion of an engagement device.

FIG. 6 is a schematic side view illustrating an example capture portion of an engagement device.

FIG. 7 is a schematic side view illustrating an example capture portion of an engagement device.

FIG. 8 is a schematic side view illustrating an example capture portion of an engagement device.

FIG. 9 is a schematic side view illustrating an example capture portion of an engagement device.

FIG. 10A is perspective view illustrating a capture portion of an example engagement device engaged with a cinch member of a prosthetic device.

FIG. 10B is a perspective view illustrating the capture portion of FIG. 10A disengaged from the cinch member.

FIG. 11 is a schematic side view illustrating an example release mechanism of an engagement device.

FIG. 12 is a schematic side view illustrating an example release mechanism of an engagement device.

FIG. 13 is a schematic side view illustrating an example release mechanism of an engagement device.

FIG. 14 is a schematic side view illustrating an example release mechanism of an engagement device.

FIG. 15 is a schematic side view illustrating an example release mechanism of an engagement device.

FIG. 16 is a schematic side view illustrating an example release mechanism of an engagement device.

FIG. 17 illustrates an example distal portion of an example engagement device including multiple capture portions.

FIG. 18 illustrates an example distal portion of an example engagement device including multiple capture portions.

FIG. 19 is a flowchart illustrating an example technique according to the present disclosure.

### DETAILED DESCRIPTION

This disclosure describes a medical system including a prosthetic device. The prosthetic device may be, for example, a prosthetic heart valve device configured to operate as a heart valve within a heart. The prosthetic device is configured to establish a relatively compact delivery configuration for delivery to a heart of a patient. The prosthetic device is configured such that a clinician may cause the prosthetic device to expand from the delivery configuration to an expanded configuration when the prosthetic device is positioned in proximity to a native heart valve, such that the prosthetic device engages tissue of the heart to position a valve assembly substantially within an annulus of the native heart valve. The prosthetic device includes a cinch member configured to aid in delivery, deployment, and/or recapture of the prosthetic device within the patient, such that a clinician may position, adjust, recapture, and/or deploy the prosthetic device during an implantation procedure before disengaging the prosthetic device from an engagement device (e.g., a portion of a delivery system).

In some examples, the cinch member is configured to enable disengagement from the engagement device (e.g., the delivery system) in a manner limiting and/or substantially eliminating forces imparted to the implanted prosthetic device. Limiting and/or substantially eliminating forces on the prosthetic device during disengagement may reduce a risk of dislodging and/or mispositioning the prosthetic device during the disengagement. In examples, and if necessary, the cinch member enables extraction of the prosthetic device from the patient. For example, the cinch member may be configured to reduce a size of an at least partially expanded prosthetic device to enable extraction from the patient.

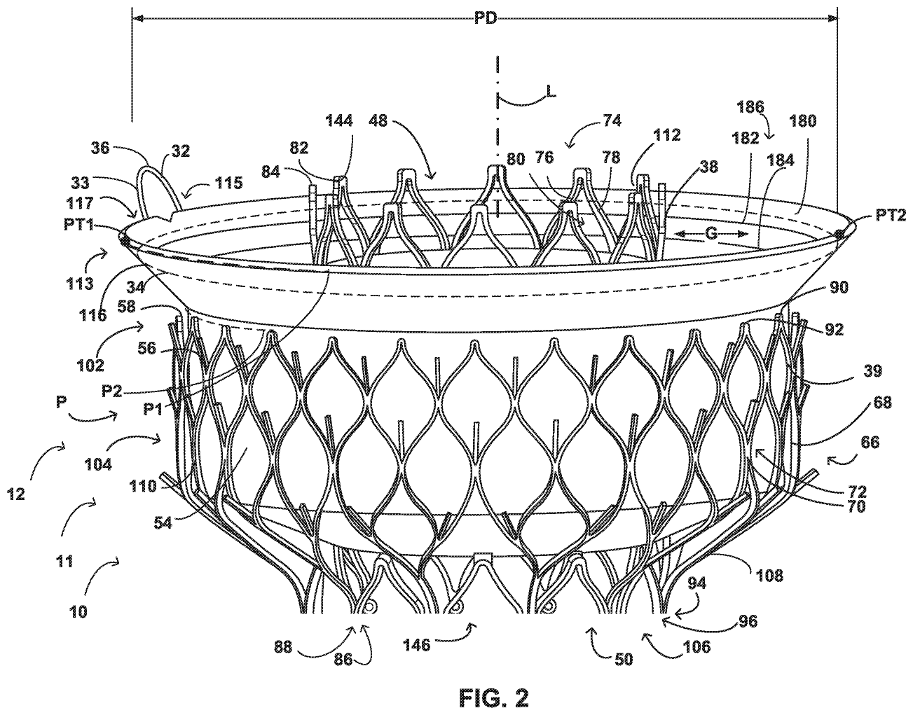

The cinch member is sized to remain as a portion of the prosthetic device following implantation of the prosthetic device within the heart. In examples, the cinch member defines a closed loop configured to reduce a perimeter dimension (e.g., a diameter) of a device perimeter defined by the prosthetic device. The device perimeter may be defined by, for example, an outer support of the prosthetic device, such as an anchoring member and/or a brim substantially surrounding an axis of the prosthetic device. In some examples, the cinch member is configured to control and/or reduce the perimeter dimension (e.g., perimeter dimension PD (FIG. 2)) of the device perimeter such that an expanded size of the prosthetic device during placement and implantation can be controlled (e.g., by a clinician).

The perimeter dimension may be a length defined by the device perimeter of the prosthetic device. For example, when the prosthetic device defines a substantially circular (e.g., circular or nearly circular to the extent permitted by manufacturing tolerances) device perimeter, the perimeter dimension may be a diameter or other chord (e.g., a nondiametrical chord) between two or more points on the device perimeter. In examples, the perimeter dimension is a length of one or more line segments extending from a first point on the on the device perimeter to a second point on the device perimeter. The one or more line segments may be a single line segment or two or more joined line segments. A line segment may be a straight line segment, a curved line segment, a curvilinear line segment, or another type of line segment. The prosthetic device may define a perimeter having any shape, such as at least a portion of a substantially circular shape, elliptical shape, oval shape, polygonal shape, or other shape.

In some examples, the cinch member is configured such that a force imparted to the cinch member (e.g., a pulling force) by an engagement device positioned (e.g., by a clinician) within a heart chamber causes a reduction in the perimeter dimension and a reduction in the size of the prosthetic device. The cinch member may be configured such that, when the engagement member is engaged with the cinch member, movement of the engagement device toward the cinch member allows the prosthetic device to expand and increase the size of the prosthetic device within the heart chamber. Hence, the cinch member is configured to allow a clinician to control a size defined by the prosthetic device during an implantation procedure. The cinch member is configured to limit forces imparted to the implanted prosthetic device by the engagement device as the engagement device is disengaged (e.g., by the clinician).

Retaining the cinch member as a portion of the prosthetic device following implantation avoids a need to retract a temporary suture which might otherwise be required to control (e.g., by cinching) the size of the prosthetic device when the prosthetic device is positioned (e.g., by a clinician) within a heart chamber. Removal of such a temporary suture from the patient following implantation may impart forces to the implanted prosthetic device, potentially altering and/or otherwise impacting the implanted position of the prosthetic device. The cinch member disclosed herein avoids the need for removal of such temporary sutures, limiting impacts to the implanted prosthetic device. Additionally, the systems according to the present disclosure may remove steps from the loading procedure, which may save time and thus improve efficiency of the loading process. Prosthetic devices and systems according to the present disclosure may find application in, for example, delivery of many different medical devices including heart valve prosthesis (e.g., prosthetic mitral valve, tricuspid valve, aortic valve, or any native heart valve), self-expanding grafts and scaffolds, or occlusion devices as examples.

The prosthetic device is configured to reside with a heart of a patient to, for example, act in place of a native heart valve within the heart. The prosthetic device includes an outer structure configured to engage an annulus of a native heart valve to help secure the prosthetic device in an annulus of the native heart valve. For example, the outer structure may include an anchoring member configured to engage the annulus. The anchoring member may be configured such that a valve axis defined by the prosthetic device passes through the annulus when the anchoring member engages the annulus. In examples, the outer support includes a valve brim mechanically supported by the anchoring member. The valve brim may be configured to position substantially upstream of the annulus when the anchoring member engages the annulus. In some examples, the valve brim may be configured to act as an imaging marker to assist a clinician in positioning the prosthetic device.

The prosthetic device may include a valve support mechanically supported by the outer support and surrounding the valve axis. The valve support may be configured to cause a valve assembly to position within or in the vicinity of the annulus when the outer structure grips the annular wall. The valve assembly may be configured to allow blood to flow along a flow path from an inflow region of the prosthetic device to an outflow region of the prosthetic device. In some examples, the valve axis passes through the valve assembly. In some examples, the prosthetic device (e.g., the outer support, valve support, or another portion of the prosthetic device) defines the device perimeter substantially conforming to a path over which the cinch member defines a closed loop. In examples, the device perimeter surrounds the valve axis. In some examples, the perimeter defined by the prosthetic device is substantially perpendicular to the valve axis (e.g., when the prosthetic device is in the expanded configuration).

In some examples, the cinch member includes a body (“member body”) forming a closed loop, such as, for example, a suture material configured to define a closed loop. The cinch member may be configured to define the closed loop (e.g., around the valve axis) when the prosthetic device is in the delivery configuration and/or in the expanded configuration. As used herein, when the cinch member defines a closed loop, this may mean the cinch member (e.g., the member body) includes a portion defining a closed path. The closed path may be, for example, a substantially circular-shaped path, a substantially ovular-shaped path, or some other closed path.

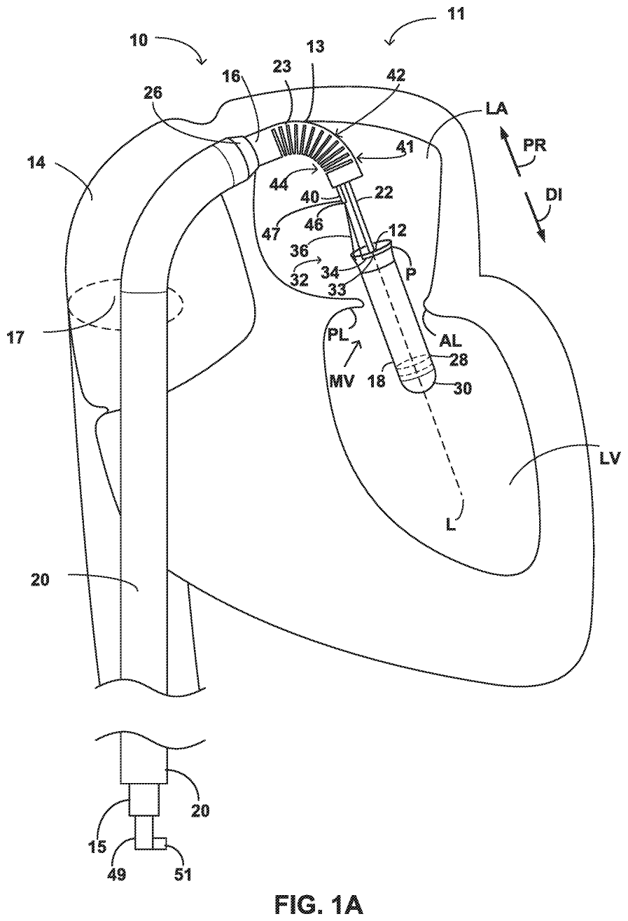

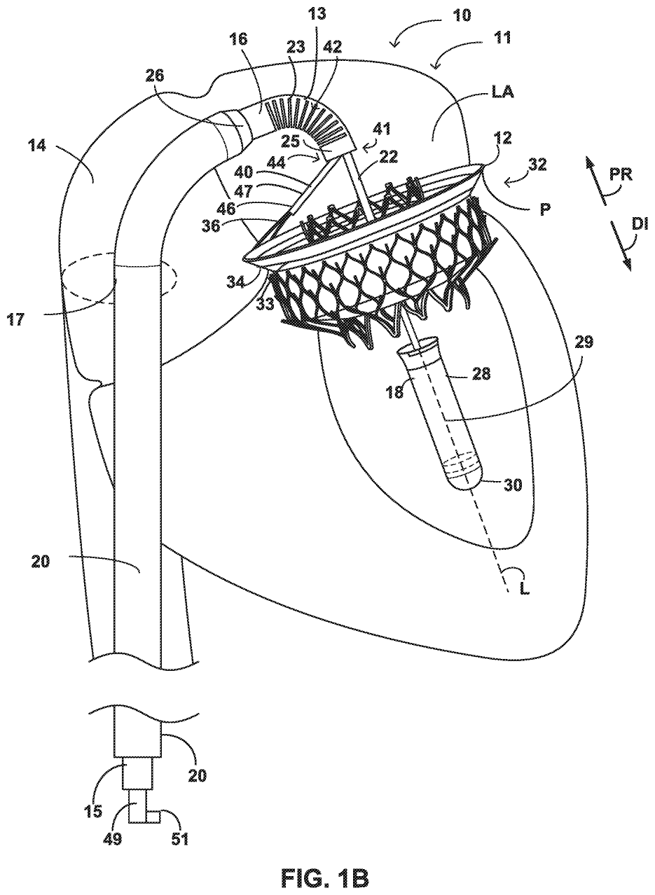

In examples, the prosthetic device is configured to define a first perimeter dimension in the delivery configuration and a second perimeter dimension in the expanded configuration, wherein the second perimeter dimension is greater than or equal to the first perimeter dimension. In examples, the prosthetic device defines the first perimeter dimension when the prosthetic device is at least partially enclosed by a capsule housing of a delivery capsule (e.g., capsule housing 28 of delivery capsule 18 (FIG. 1A)). The cinch member is configured such that a clinician may increase and/or decrease the second perimeter dimension (e.g., using an engagement device engaged with the cinch member) as the prosthetic device radially expands from the delivery configuration.

In examples, the member body includes a first portion (“first body portion”) and a second portion (“second body portion”) defining the closed loop. The member body may be configured such that the first body portion resides within one or more passages defined by the prosthetic device around the device perimeter. The second body portion may be a portion of the member body residing substantially outside the one or more passages and/or otherwise accessible to an engagement device. The first body portion may be configured to slidably translate within the one or more passages, such that when the engagement device engages and exerts a force on the second body portion in a direction away from the prosthetic device (e.g., pulls on the second body portion), the first portion translates within the one or more passages around the device perimeter to substantially cinch the prosthetic device, causing a reduction in the size of the defined perimeter. The cinching of the prosthetic device may reduce a radial displacement of the prosthetic device (e.g., reduce a displacement defined by the prosthetic device substantially perpendicular to the valve axis).

In some examples, the first body portion and/or the second body portion are any suitable portion of the member body, such that portions of the member body defining the first body portion and/or second body portion may be dependent on an orientation of the member body with respect to the outer support, valve support, and/or some other portion of the prosthetic valve device. For example, the member body may include a first section defining the first body portion and a second section defining the second body portion when the member body has a first orientation with respect to the outer support, valve support, and/or some other portion of the prosthetic valve device. The member body may be configured such that, when the member body has a second orientation different from the first orientation, the first section at least partially defines the first body portion and the second section at least partially defines the second body portion. Thus, in some examples, the sections of the member body defining the first portion and/or the second portion are substantially independent of the orientation of the member body with respect to the outer support, valve support, and/or some other portion of the prosthetic valve device.

In some examples, the member body is configured to limit an ability of the first section to define the second section and/or limit an ability of the second section to define the first portion. For example, the member body may be configured such that the second section defines the second portion in a manner which increases the accessibility of the second portion to the engagement device and/or assists the engagement device in exerting the force on the second portion.

The one or more passages defined by the prosthetic device may be configured to substantially contain some portion of the cinch member (e.g., the first body portion) while enabling the cinch member to cinch and relax about the device perimeter, depending on the force exerted on the cinch member (e.g., exerted on the second portion) by an engagement device. The one or more passages may be defined by one or more portions of the prosthetic valve device, such as one or more portions of the outer support (e.g., the brim and/or anchoring member), the valve support, and/or another portion of the prosthetic valve device. A given passage may be a volume configured to allow at least a first portion of the member body to pass therethrough. The prosthetic device may define a first opening (“first passage opening”) opening to the given passage and a second opening (“second passage opening”) opening to the given passage, such that the member body may pass through the first passage opening and the second passage opening when the member body slidably translates within the passage. In examples, the prosthetic device may define the one or more passages substantially around the device perimeter surrounded by the member body of the cinch member.

In examples, the medical system includes the engagement device configured to engage the second body portion of the member body. The engagement device may be a component of a delivery system configured to deliver the prosthetic device to a heart or other anatomical location of the patient for implantation, and configured to be removed from the patient following the implantation. The cinch member is configured as a component of the prosthetic device. Stated similarly, the prosthetic device may include a substantially permanent cinch member which remains attached to the prosthetic device post-operation, while the engagement device is configured to be extracted from the patient. In some examples, the second body portion may be defined by a second length of the cinch member (e.g., the member body) joined to a first length defining the first body portion. The first length and the second length may define the closed loop of the cinch member.

Specific examples are now described with reference to the figures. Unless otherwise indicated, the terms “distal” and “proximal” are used in the following description with respect to a position relative to a treating clinician. “Distal” or “distally” are positions distant from or in a direction away from the clinician, and “proximal” and “proximally” are positions near or in a direction toward the clinician.

The following detailed description is merely exemplary in nature and is not intended to be limiting. Although the description of examples is in the context of prosthetic heart valves, prosthetic devices according to the present disclosure may be other medical devices. Although delivery systems and techniques are described for delivering a heart valve prosthesis within a heart, the delivery systems and techniques described may be used for other medical devices delivered to other parts of the body.

FIG. 1A illustrates a medical system 10 including a portion of an example delivery system 11 for delivering an example prosthetic heart valve device 12 (“device 12”) to a target site within a heart 14. Delivery system 11 includes a delivery catheter 16 supporting a delivery capsule 18, with delivery catheter 16 extending from a lumen defined by a guide catheter 20. FIG. 1A illustrates device 12 in a delivery configuration within delivery capsule 18, where delivery capsule 18 constrains device 12 against radial expansion. FIG. 1B illustrates delivery system 11 with device 12 an expanded configuration, where delivery capsule 18 has been displaced from device 12 (e.g., by a clinician via delivery catheter 16) such that capsule 18 provides limited or substantially no constraint on the radial expansion of device 12.

For the purpose of illustration, FIG. 1A and FIG. 1B illustrate delivery system 11 positioning device 12 in a native mitral valve MV of heart 14 using a trans-septal delivery approach. In some examples, device 12 may be configured to be placed in a native heart valve (e.g., a tricuspid valve, an aortic valve, or a pulmonary valve) using a transcatheter percutaneous delivery system. Other approaches may be utilized, such as a trans-apical delivery approach.

Referring to FIG. 1A, guide catheter 20 is positioned in a trans-septal opening 17 to provide access to the left ventricle LV. Delivery catheter 16 extends through guide catheter 20 such that a distal portion 25 of a catheter body 23 projects beyond a distal end 26 of guide catheter 20. Capsule 18 may then be positioned between a posterior leaflet PL and an anterior leaflet AL of mitral valve MV. Using a control unit (not pictured), catheter body 23 may be moved in the distal direction (as indicated by arrow DI), the proximal direction (as indicated by arrow PR), and/or rotated along a longitudinal axis of catheter body 23 to position capsule 18 at a desired location and orientation within the opening of mitral valve MV (or another native heart valve within heart 14).

At a target location (e.g., proximate to an annulus of a native heart valve), capsule 18 (e.g., capsule housing 28) may be at least partially displaced (e.g., in the distal direction DI) to deploy device 12 from capsule 18. Delivery system 11 may be configured such that displacement of capsule housing 28 from device 12 allows device 12 to expand to the expanded configuration. In some examples, capsule 18 may be open-ended at the distal end of capsule housing 28. In some examples, capsule 18 may be configured to circumferentially surround at least part of device 12.

Delivery system 11 may include deployment member 29 configured to displace capsule housing 28 (e.g., cause a displacement of capsule housing 28 from distal portion 25 of catheter body 23) and thus displace delivery capsule 18. Deployment member 29 may extend into capsule housing 28 (as illustrated using dashed lines) At or proximate to the target location, device 12 may be deployed from capsule 18 by driving deployment member 29 in a distal direction (e.g., further into the left ventricle LV) to move capsule housing 28 and/or end cap 30 distally (e.g., further into the left ventricle LV), such that capsule housing and/or end cap 30 displaces from distal portion 25 of catheter body 23. In some examples, device 12 may be deployed from capsule 18 by moving distal portion 25 proximally. As device 12 exits capsule housing 28, device 12 be held in the initial delivery configuration or a partially expanded configuration by engagement device 40. Subsequently, translation of engagement device 40 in a distal direction may allow device 12 to expand radially to secure device 12 in an annulus of a native heart valve (e.g., the annulus of mitral valve MV).

Device 12 is configured to define a valve axis L and a device perimeter P substantially surrounding valve axis L. Device 12 is configured such that a radial expansion of device 12 (e.g., from the delivery configuration to the expanded configuration) increases a perimeter dimension defined by device perimeter P. In similar manner, device 12 is configured such that a radial contraction of device 12 (e.g., from the expanded configuration to the delivery configuration) decreases a perimeter dimension defined by device perimeter P. Device perimeter P may be defined by any portion of device 12. In examples, an outer support (e.g., outer support 39 (FIGS. 2, 3A, 3B, 4)) defines device perimeter P.

As will be discussed further, device 12 includes cinch member 32. Cinch member 32 is configured to control and/or reduce the perimeter dimension defined by device perimeter P, such that an expanded size of device 12 during placement and implantation may be controlled (e.g., by a clinician). Cinch member 32 may be, for example, a suture or some other material configured to define a closed loop. In some examples, cinch member 32 may comprise a polymer fabric material. In some examples, cinch member 32 may comprise an ultrahigh molecular weight polyethylene.

Cinch member 32 includes a member body 33 defining a first body portion 34 and a second body portion 36. Cinch member 32 is configured such that, when an engagement device 40 engages and exerts a force on second body portion 36 in a direction away from device 12 (e.g., pulls on second body portion 36), first body portion 34 translates to substantially cinch device 12, causing a reduction in the size of device perimeter P (e.g., a reduction in a radial displacement defined by perimeter P). In examples, device 12 may be resiliently biased to cause a radial expansion of device 12 (e.g., to engage an annulus of a heart valve such as the MV). Cinch member 32 may be configured to overcome the resilient bias to control and/or reduce the perimeter dimension of perimeter P when engagement device 40 exerts the force on second body portion 36. Hence, cinch member 32 may be configured such that a clinician may utilize engagement device 40 to cause cinch member 32 to constrain the radial expansion of device 12 during, for example, an implantation procedure. The clinician may cause engagement device 40 to release cinch member 32 when device 12 is in position within heart 14 (e.g., within the annulus of the MV), such that engagement device 40 ceases to exert the force on second body portion 36. Engagement device 40 (e.g., when disengaged from second body portion 36) may be withdrawn from heart 14 as cinch member 32 remains with device 12. Disengagement of engagement device 40 and second body portion 36 may thus limit and/or substantially avoid imparting forces to device 12 when device 12 is in an implantation position within heart 14.

In examples, delivery catheter 16 defines lumen 41. Engagement device 40 may be configured to translate within lumen 41. In some examples, lumen 41 is at least one of a main lumen 42 or an auxiliary lumen 44. In some examples, auxiliary lumen 44 may contain engagement device 40. Alternatively, in some examples, engagement device 40 may be contained within main lumen 42. Engagement device 40 includes a capture portion 46 configured to engage device 12. In examples, delivery catheter 16 includes a distal portion 13 (“delivery catheter distal portion 13”) configured to be positioned (e.g., by a clinician) intracorporeal to a patient and a proximal portion 15 (“delivery catheter proximal portion 15”) configured to remain extracorporeal to the patient. Engagement device 40 may include a distal portion 47 (“engagement device distal portion 47”) configured to be positioned (e.g., by a clinician, via delivery catheter 16) intracorporeal to the patient and a proximal portion 49 (“engagement device proximal portion 49”) configured to remain extracorporeal to the patient. Engagement device distal portion 47 may include capture portion 46. In examples, engagement device proximal portion 49 includes a release mechanism 51 configured to cause capture portion 46 to engage with and/or disengage from device 12. Release mechanism 51 may be manipulated and/or controlled by a clinician to cause capture portion 46 to engage with and/or disengage from device 12. Engagement device 40 may be configured such that movement of engagement device proximal portion 49 relative to delivery catheter proximal portion 15 (e.g., via translation of engagement device 40 through lumen 41) causes movement of engagement device distal portion 47 relative to delivery catheter distal portion 13. Hence, delivery system 11 may be configured such that a clinician may cause engagement and/or disengagement of capture portion 46 with device 12 and/or cause movement of engagement device distal portion 47 relative to delivery catheter distal portion 13 when device 12 and engagement device 40 are positioned within heart 14.

Engagement device 40 to may be configured to translate within lumen 41 to control (e.g., via cinch member 32) a perimeter dimension defined by perimeter P of device 12. For example, when engaged with second body portion 36, engagement device 40 may translate substantially in the distal direction DI to cause cinch member 32 to allow device 12 to radially expand to increase a perimeter dimension defined by perimeter P. Engagement device 40, when engaged with second body portion 36, may translate substantially in the proximal direction PR to cause cinch member 32 to radially contract device 12 to radially reduce the perimeter dimension defined by perimeter P. In examples, second body portion 36 is engaged with capture portion 46 of engagement device 40 during deployment from delivery capsule 18, such that a clinician may control the radial displacement of device 12 during the deployment. In some examples, engagement device 40 may be configured to flex away from valve axis L to allow device 12 to completely expand to the delivery configuration. In some examples, delivery system 11 includes delivery catheter 16 defining main lumen 42 and auxiliary lumen 44, and deployment member 29 may be configured to translate within main lumen 42 while engagement device 40 may be configured to translate within auxiliary lumen 44.

Control of the perimeter dimension using engagement device 40 may allow a clinician to control the deployment of device 12 from the delivery configuration (FIG. 1A) to the expanded configuration (FIG. 1B). In some examples, device 12 may be resiliently biased to take on the expanded configuration (FIG. 1B). Engagement device 40 may exert a force on second body portion 36 of cinch member 32 to maintain device 12 in the initial delivery configuration, and translation of engagement device 40 in the distal direction may allow first body portion 34 of cinch member 32 to relax about a perimeter of device 12, allowing device 12 to transition to the deployment configuration when the engagement device 40 slidably translates in a distal direction along valve axis L toward first body portion 34, as illustrated in FIG. 1B. As engagement device 40 translates in a distal direction, the perimeter dimension (and device 12) may assume a series of dimensions between the delivery configuration (FIG. 1A) and the expanded configuration (FIG. 1B).

Conversely, cinch member 32 may be configured to transition back into the delivery configuration when engagement device 40 slidably translates in a proximal direction along valve axis L, by causing first body portion 34 to cinch a device perimeter of device 12. In some examples, device 12 may be resiliently biased to be configured in the expanded configuration of FIG. 1B, and cinch member 32 may be configured to overcome the resilient bias to reduce a perimeter dimension of device 12 when engagement device 40 exerts a force on second body portion 36 in a direction away from first body portion 34, maintaining or returning device 12 to the reduced diameter initial delivery configuration of FIG. 1A. In some examples, device 12 including cinch member 32 may allow for a tapered shape on the proximal end of device 12, which may be desirable to recover device 12 through the vasculature of a patient.

The examples provided are described herein with reference to devices, systems, and methods for positioning and deploying a prosthetic heart valve to a native mitral valve or tricuspid valve. However, other applications and other examples in addition to those described herein are within the scope of the present technology. For example, at least some examples of the present technology may be useful for delivering and deploying prosthetics to other native valves such as the aortic valve or pulmonary valve.

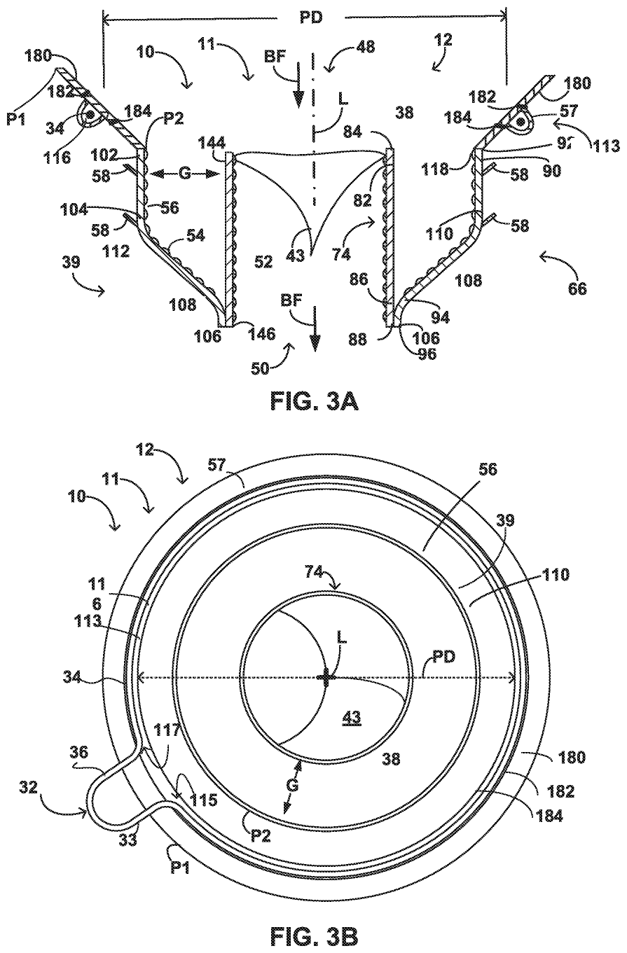

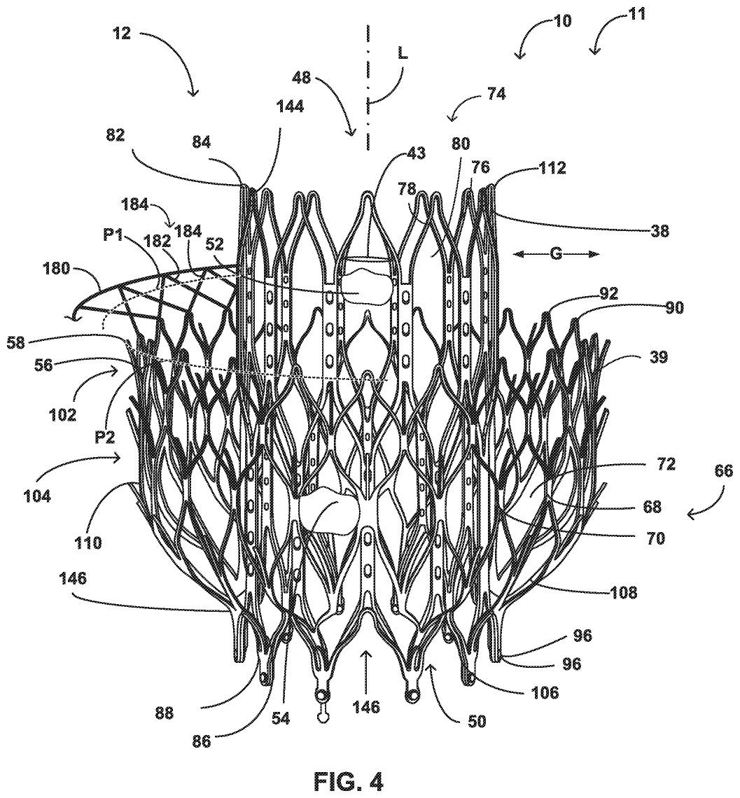

FIG. 2 illustrates a perspective view of portions of device 12 in an expanded configuration. FIG. 3A is a cross-sectional view taken along valve axis L defined by device 12. FIG. 3B is a top plan view of device 12 in an expanded configuration. FIG. 4 is a perspective view similar to FIG. 2, with some elements of device 12 removed such that only the underlying structure is illustrated.

Device 12 includes a valve support 38, outer support 39 attached to the valve support 38, and a prosthetic valve assembly 43 (“valve assembly 43”) within the valve support 38. Outer support 39 may be coupled to (e.g., attached) to valve support 38. Valve assembly 43 may be coupled to valve support 38. In examples, valve support 38 mechanically supports valve assembly 43. In some examples, device 12 defines a valve axis L intersecting valve assembly 43. In some examples, valve support 38 and/or outer support 39 surround some portion of valve axis L.

In examples, device 12 (e.g., outer support 39) is configured to expand radially outward from a delivery configuration (e.g., when housed within delivery capsule 18 (FIG. 1A)) to the expanded configuration depicted in FIG. 1B, FIGS. 2-3B, and 4. In examples, outer support 39 includes an anchoring member 56 and valve brim 180. Device 12 may be configured to expand radially outward to substantially grip an annulus of a native heart valve when, for example, device 12 transitions from the delivery configuration to the expanded configuration. Device 12 (e.g., anchoring member 56) may be configured to expand radially to accommodate (e.g., substantially conform to) a perimeter of the annulus of a native heart valve when device 12 is in the expanded configuration. In examples, outer support 39 (e.g., anchoring member 56 and/or valve brim 180) is configured to expand radially outward from valve support 38 to establish a gap G between outer support 39 and valve support 38. Gap G may define a radial displacement relative to valve axis L. In some examples, the radial displacement is substantially perpendicular to valve axis L. In some examples, outer support 39 is configured to define an anchoring delivery radius relative to valve axis L when device 12 is in the delivery configuration and an anchoring expanded radius relative to valve axis L when device 12 is in the expanded configuration, and the anchoring delivery radius is less than the anchoring expanded radius.

Device perimeter P at least partially surrounds valve axis L. Device 12 may define device perimeter P around any portion of device 12 and/or at least partially around any portion of device 12. In examples, the perimeter is a closed boundary extending around some portion of device 12. For example, device 12 may define a device perimeter P such as perimeter P1, perimeter P2, or another device perimeter P defined by device 12. Perimeter P1, P2 may be defined by any portion of device 12. For example, perimeter P1 may be defined by some portion of brim 180, such as a portion of outer support sealing member 54 comprising brim 180 and/or one or more of brim structures 186. Perimeter P2 may be defined by some portion of anchoring member 56, such as a portion of outer support sealing member 54 comprising anchoring member 56 and/or one or more outer support struts 66 (FIG. 4). In examples, device perimeter P (e.g., perimeter P1, P2) surrounds valve axis L. In some examples, device perimeter P is substantially perpendicular to the valve axis L (e.g., when the prosthetic device is in the expanded configuration). In some examples, device perimeter P may be an outer perimeter (along an outer radial surface of device 12). In some examples, the defined perimeter is a substantially planar perimeter, such that the perimeter substantially lies within a geometric plane (e.g., lies in the geometric plane or nearly fully lies in the geometric plane to the extent permitted by manufacturing tolerances). In some examples, the defined perimeter is a nonplanar perimeter, such that portions of the defined perimeter may lie in different geometric planes. In examples, device perimeter P (e.g., perimeter P1, P2) surrounds valve axis L. In some examples, the defined perimeter P is substantially perpendicular to valve axis L.

FIGS. 2-4 illustrate device 12 in an expanded configuration, Device 12 supports cinch member 32. Cinch member 32 is configured to control (e.g., reduce) a device perimeter P defined by device 12, such as a perimeter P1 defined by a brim 180 and/or a perimeter P2 defined by an anchoring member 56. Cinch member 32 includes first body portion 34 contained within one or more passages 113 defined by device 12, such as passage 116. In FIG. 2, first body portion 34 is illustrated within passage 116 as a dashed line. Passage 116 may be defined by one or more portions of the device 12, such as one or more portions of the outer support 39 (e.g., brim 180 and/or anchoring member 56), valve support 38, and/or another portion of the prosthetic valve device. A given passage of the one or more passages 113 (e.g., passage 116) may be a volume configured to allow at least some portion of first body portion 34 of member body 33 to pass therethrough. In examples, device 12 defines first passage opening 115 opening to passage 116 and a second passage opening 117 opening to passage 116, such that the member body 33 may pass through first passage opening 115 and second passage opening 117 when member body 33 slidably translates within passage 116. In examples, device 12 is configured such that member body 33 surrounds valve axis L when member body 33 resides within the one or more passages 113 (e.g., passage 116). Any portion of member body 33 may define first body portion 34, and any other portion of member body 33 may define second body portion 36.

Device 12 may define any number of passages configured such that cinch member 32 (e.g., member body 33) may control (e.g., reduce) a perimeter dimension of a perimeter P (e.g., perimeter P1, perimeter P2) when cinch member 32 (e.g., first body portion 34 translates within the one or more passages 113. In some examples, the one or more passages 113 (e.g., passage 116) may be a single passage (e.g., a single passage at least partially surrounding valve axis L) such as passage 116. In some examples, the one or more passages 113 may be a plurality of passages which includes passage 116 and one or more additional passages. Device 12 may be configured such that cinch member 32 (e.g., member body 33) may control (e.g., reduce) a perimeter dimension of a perimeter P (e.g., perimeter P1, perimeter P2) when cinch member 32 (e.g., first body portion 34) translates within passage 116 and the one or more additional passages. In addition to translation through passage 116 and/or the one or more additional passages, member body 33 may be configured to reside (e.g., remain substantially stationary with respect to device 12) within passage 116 and/or the one or more additional passages. For example, member body 33 may be configured to reside within passage 116 and/or the one or more additional passages in the absence of a force exerted on second body portion 36 (e.g., by engagement device 40). Any of, or each of, the one or more additional passages may be configured similarly to passage 116.

Device 12 is configured such that cinch member 32 may control (e.g., reduce) a perimeter dimension PD defined by perimeter P (e.g., perimeter P1 and/or perimeter P2). In examples, device perimeter P defines a perimeter dimension PD relative to valve axis L. In examples, device perimeter P is displaced from valve axis L in a direction substantially perpendicular to valve axis L. In some examples, as illustrated, valve brim 180 defines device perimeter P, and thus defines perimeter dimension PD. Cinch member 32 defines a closed loop configured to reduce the perimeter dimension PD of perimeter P defined by valve brim 180, and the displacement of valve brim 180 may be controlled by a clinician by cinch member 32. For example, cinching of cinch member 32 may cause device perimeter P to move closer to valve axis L, reducing perimeter dimension PD. Similarly, relaxation of cinch member 32 may cause device perimeter P to move further from valve axis L, increasing perimeter dimension PD.

In some examples, perimeter dimension PD is a length defined by device perimeter P1, P2 of device 12. For example, in examples, where device perimeter P1, P2 is a substantially circular device perimeter, perimeter dimension PD may be a diameter or other chord between two or more points on device perimeter P1, P2. In examples, perimeter dimension PD is a length of one or more line segments extending from a first point (e.g., PT1) on the device perimeter (e.g., perimeter P1) to a second point (e.g., PT2) on the device perimeter. Although illustrated in FIG. 2, FIG. 3A, and/or FIG. 3B as a single, substantially straight line segment, the one or more line segments may be a single line segment or two or more joined line segments, including one or more straight line segments, curved line segments, curvilinear line segments, and/or another types of line segments. Further, although illustrated in FIG. 2, FIG. 3A, FIG. 3B, and/or FIG. 4 as a substantially circular or oval-shape perimeter, device perimeter P1, P2 may define any shape or portion of any shape, such as at least a portion of a substantially circular shape, elliptical shape, oval shape, polygonal shape, or other shape.

In examples, cinch member 32 may define a closed loop around valve axis L. In examples, device perimeter P defined by device 12 surrounds valve axis L. In some examples, cinch member 32 defines a first radial displacement from valve axis L and device 12 defines device perimeter P such that device perimeter P defines a second radial displacement from cinch member 32. The first radial displacement and/or the second radial displacement may be perpendicular to valve axis L. The first radial displacement may be greater than, less than, or substantially equal (e.g., to the extent permitted by manufacturing tolerances) to the second radial displacement.

Passage 116 may be configured such that displacement of second body portion 36 in a direction substantially away from valve axis L (e.g., displacement caused by engagement device 40) causes first body portion 34 to translate within passage 116 around device perimeter P to substantially cinch device 12, causing a reduction in the perimeter dimension PD defined by perimeter P. The reduction in the perimeter dimension PD may reduce a radial displacement of the device 12 (e.g., reduce a displacement defined by device 12 substantially perpendicular to the valve axis L). Similarly, passage 116 may be configured such that a force exerted on second body portion 36 (e.g., exerted by engagement device 40) substantially prevents the translation of first body portion 34 within passage 116 and resists any radially outward resilient biasing of outer support 39, substantially limiting and/or preventing an increase in the perimeter dimension PD defined by perimeter P. Substantially limiting and/or preventing an increase in the perimeter dimension PD may substantially limiting and/or prevent increases the radial displacement of the device 12 (e.g., while positioning device 12 during an implantation procedure.).

Cinch member 32 may be configured to control and/or limit the Gap G in a manner similar to the manner described above with respect to perimeter dimension PD. For example, gap G may be reduced by cinching of cinch member 32 about device perimeter P. For example, when device perimeter P is defined by anchoring member 56 or valve brim 180, cinching of cinch member 32 may cause outer support 39 to move closer to valve support 38. Similarly, relaxation of cinch member 32 may gap G to increase as relaxation of cinch member 32 allows outer support 39 to move further from valve support 38.

In examples, valve support 38 is a substantially rigid member configured to retain its shape (e.g., define a substantially constant radius around valve axis L) when outer support 39 expands radially outward. In some examples, valve support 38 may be configured to expand radially outward when outer support 39 expands radially outward. In some examples, valve assembly 43 may be configured to expand radially when valve support 38 and/or outer support 39 expands radially. In some examples, outer support 39 and/or valve support 38 may be resiliently biased to displace in a direction away from valve axis L (e.g., to establish the expanded configuration). In some examples, device 12 is resiliently biased to resist the displacement of the outer support 39 toward valve axis L.

Valve support 38 includes an inflow region 144 and an outflow region 146. In examples, device 12 defines a downstream direction (e.g., the direction of the arrows BF) extending from the inflow region 144 toward the outflow region 146 and an upstream direction (e.g., substantially opposite the direction of arrows BF) extending from outflow region 146 toward inflow region 144. The downstream direction and/or upstream direction may be substantially parallel to valve axis L. In examples, inflow region 144 defines an inlet 48 of valve support 38 (“valve support inlet 48”). Outflow region 146 may define an outlet 50 of valve support 38 (“valve support outlet 50”). In examples, device 12 is configured to define a flow path for blood flow (BF) from valve support inlet 48 to valve support outlet 50.

Valve assembly 43 is configured to be supported within the valve support 38 to allow blood to flow through device 12 in the downstream direction (e.g., from valve support inlet 48 to valve support outlet 50). In examples, valve assembly 43 is configured to allow blood flow through device 12 in the downstream direction, but limit and/or substantially prevent blood flow through device 12 in the upstream direction. In examples, device 12 is configured such that valve assembly 43 controls substantially all blood flow through device 12 when device 12 is in the expanded configuration and positioned within an annulus of a native heart valve.

For example, device 12 may include a valve support sealing member 52 configured define the flow path through valve support 38. Valve support sealing member 52 may be configured to substantially direct blood flow from valve support inlet 48 to valve support outlet 50. In examples, valve support sealing member 52 is configured to limit and/or substantially prevent a flow of blood through valve support 38 in a radial direction away from valve axis L. Device 12 may include an outer support sealing member 54 configured to limit and/or substantially prevent a flow of blood through outer support 39. Device 12 may be configured such that, when device 12 is in the expanded configuration and positioned within an annulus of a native heart valve, valve support sealing member 52 and outer support sealing member 54 cause substantially all blood flow (e.g., all or nearly all to the extent permitted by the material from which sealing members 52, 54 are formed) passing through device 12 to flow through valve assembly 43. In examples, passage 116 may be defined by valve support sealing member 52 and/or outer support sealing member 54. In some examples, first body portion 34 may be formed from or be a portion of valve support sealing member 52 or outer support sealing member 54.

In examples, when device 12 is in the expanded configuration and positioned within an annulus of a native heart valve, valve support sealing member 52, outer support sealing member 54, and valve assembly 43 allow blood flow through device 12 in the downstream direction (e.g., from inflow region 144 to outflow region 146), while limiting and/or substantially preventing blood flow through device 12 in the upstream direction e.g., from outflow region 146 to inflow region 144). Device 12 may be configured such that, when device 12 is in the expanded configuration and positioned within an annulus of a native heart valve, inflow region 144 is fluidically coupled to a first chamber (e.g., an atrium) of a heart 14 (FIG. 1A) and outflow region 146 is fluidically coupled to a second chamber (e.g., a ventricle) of a heart 14 (FIG. 1A).

Outer support 39 of device 12 includes anchoring member 56 configured to engage with tissue to help secure device 12 in a heart of a patient. For example, anchoring member 56 can be configured to grip an annular wall of a native heart valve when device 12 is positioned within an annulus of the native heart valve and device 12 is in the expanded configuration. Anchoring member 56 may be configured to secure device 12 within the annulus of the native heart valve, such that valve assembly 43 may allow blood to flow from inflow region 144 through outflow region 146. Device 12 may be configured to cause valve assembly 43 to substantially position within or in the vicinity of the annulus when anchoring member 56 grips the annular wall, such that valve assembly 43 allows blood to flow along a flow path substantially parallel to valve axis L (e.g., from inflow region 144 to outflow region 146). In examples, anchoring member 56 includes a plurality of anchoring elements 58 (e.g., barbs, hooks, cleats, tines, or other elements) configured to engage tissue of heart 14. In examples, anchoring elements 58 may be configured to engage an annular wall of a native heart valve when device 12 is positioned within an annulus of the native heart valve and device 12 is in the expanded configuration.

First body portion 34 and second body portion 36 of cinch member 32 (e.g., member body 33) combine to define a closed loop around a device perimeter P defined by device 12 (e.g., perimeter P1, perimeter P2, or another perimeter). First body portion 34 translates through first passage opening 115 and second passage opening 117 of passage 116 defined by device 12, such that any portion of member body 33 may define first body portion 34. Cinch member 32 (e.g., member body 33) is configured such that second body portion 36 may be engaged by an engagement device (e.g., engagement device 40 of FIGS. 1A-1B), and, in response to a force exerted by engagement device 40, reduce perimeter dimension PD defined by device perimeter P (e.g., P1), such that a displacement between the device perimeter P and valve axis L decreases (e.g., decreases to a displacement less that present prior to engagement device 40 exerting the force.). In some examples, as illustrated, device perimeters (P1, P2) define a perimeter dimension substantially parallel to valve axis L.

In examples, cinch member 32 is configured to displace anchoring member 56 of outer support 39 toward the valve axis L by overcoming a resilient bias of device 12. Device 12 may be resiliently biased to urge outer support 39 toward the expanded configuration of FIG. 1B. The resilient biasing may act to displace outer support 39 (e.g., anchoring member 56 and/or brim 180) in a direction away from valve support 38 and/or valve axis L. Cinch member 32 may be configured to overcome the resilient biasing of device 12 such that the perimeter dimension PD defined by device 12 may be controlled (e.g., reduced) based on a force exerted on cinch member 32 (e.g., second body portion 36). In some examples, a force may be exerted on cinch member 32 by an engagement device (e.g., engagement device 40, FIGS. 1A and 1B).

In examples, outer support 39 may be resiliently biased to resist displacement of anchoring member 56 toward valve axis L. In other words, when a force on second body portion 36 of cinch member 32 is reduced or eliminated (e.g., device 12 is disengaged from engagement device 40, FIGS. 1A-B), first body portion 34 may relax and substantially cease constraining device perimeter P, allowing device 12 to expand (e.g., expand to the expanded configuration) due to the resilient bias of outer support 39. First body portion 34 of cinch member 32 may be configured to overcome the resilient bias when engagement device 40 (FIGS. 1A-1B) exerts a force on second body portion 36 (e.g., a pulling force on second body portion 36), causing anchoring member 56 to displace toward valve axis L to contract device 12 (e.g., transition to the delivery configuration from the expanded configuration).

In examples, the one or more passages 113 may substantially surround (e.g., surround at least a part of) the perimeter P of device 12 (e.g., perimeter P1, perimeter P2). First passage opening 115 and second passage opening 117 may be spaced apart on the device perimeter such that a pulling force on second body portion 36 causes first body portion 34 of cinch member 32 to displace the device perimeter towards the valve axis, rather than deforming the device perimeter.

One or more passages 116 may be defined by any path for member body 33 through a structure of device 12. In this way, first body portion 34 may be mechanically supported around at least a portion of the device perimeter P. In some examples, one or more passages 116 may be defined by an intermittent series of discrete support members (e.g., eyelets) positioned around the device perimeter and configured to mechanically support and position the cinch member. In the illustrated example, passage 116 substantially surrounds device perimeter P defined by perimeter P1 of valve brim 180. Additionally or alternatively, one or more passages 116 may surround a majority of perimeter P2 defined by anchoring member 56. Alternatively, one or more passages 116 may be defined by valve support 38 or another portion of device 12 defining a perimeter.

As discussed, medical system 10 may include delivery system 11. In examples, delivery system 11 includes engagement device 40 (FIGS. 1A, 1B). In some examples, engagement device 40 may allow a clinician to control perimeter dimension PD of device perimeter P during a medical procedure and/or disengage from device 12 at the conclusion of a procedure, leaving device 12 implanted in a patient.

In some examples, engagement device 40 may be configured to exert a force on second body portion 36 of cinch member 32. The force exerted by engagement device 40 may cause first body portion 34 of cinch member 32 to translate within the one or more passages 113 to control (e.g., reduce) a perimeter dimension PD defined by a device perimeter (e.g., perimeter P1, perimeter P2). In some examples, the force exerted by engagement device 40 may by generated and/or controlled by a clinician. A force generated or maintained by engagement device 40 on second body portion 36 may cause the perimeter dimension PD to alter in a direction substantially perpendicular to valve axis L. In examples, cinch member 32 may be configured to displace outer support 39 toward valve axis L when engagement device 40 exerts the force on second body portion 36. In examples, the force exerted by engagement device 40 on second body portion 36 causes displacement of second body portion 36 in a direction away from a portion of device 12 (e.g., outer support 39), causing cinch member 32 to cause a contraction of device 12 (e.g., reducing the perimeter dimension PD). In examples, the force exerted by engagement device 40 on second body portion 36 allows displacement of second body portion 36 in a direction toward the portion of device 12 (e.g., outer support 39), causing cinch member 32 to allow an expansion of device 12 (e.g., increasing the perimeter dimension PD). In some examples, the force exerted by engagement device 40 on second body portion 36 substantially maintains a position of second body 36 relative to the portion of device 12 (e.g., outer support 39), causing cinch member 32 to substantially maintain a configuration of device 12 (e.g., substantially maintain the perimeter dimension PD). In some examples, the force exerted by engagement device 40 may maintain device 12 in an initially delivery configuration (FIG. 1A). In some examples, perimeter dimension PD may be smaller in magnitude in the initial delivery configuration than perimeter dimension PD in the expanded configuration (FIG. 1B, 2, 3A, 3B, 4).

In some examples, engagement device 40 may be configured to exert the force (e.g., on second body portion 36) during a pre-implantation process, such as for example, during preparation and loading of a device 12 in delivery capsule 18. The force may cause device 12 to be maintained in an initial reduced-diameter delivery configuration (FIG. 1A). During an implantation process, with engagement device 40 engaged with cinch member 32, a clinician may cause engagement device 40 to translate in a distal direction and/or proximal direction through a lumen 41 of delivery system 11 to control the expansion of device 12 (e.g., control the perimeter dimension PD defined by device 12). For example, translation of engagement device 40 in a distal direction (e.g., substantially towards first body portion 34) may cause perimeter dimension PD defined by the device perimeter P to increase (e.g., due to a resilient biasing of outer support 39). In some examples, perimeter dimension PD may increase to a final delivery or expanded configuration of device 12. In some examples, at any point during or subsequent to the increase of the perimeter dimension PD from the initial delivery configuration to the final expanded configuration, translation of engagement device 40 in a proximal direction (e.g., substantially away from first body portion 34) may cause the perimeter dimension PD defined by device perimeter P to decrease (e.g., due to the force exerted on second body portion 36 by engagement member 40). In some examples, reducing perimeter dimension PD may include reducing perimeter dimension PD from an initial perimeter dimension (such as perimeter dimension PD of FIG. 3A) to a subsequent perimeter dimension PD, and the subsequent perimeter dimension PD may be less than the initial perimeter dimension PD. In this way, delivery system 11 may allow a clinician to expand and/or contract device 12 one or more times during an implantation process by translating engagement device 40 within lumen 41 to cause cinch member 32 to cinch or relax about at least a portion of device perimeter P.

Engagement device 40 may be configured such that the clinician may cause engagement device 40 to disengage from device 12 (e.g., once device 12 is positioned within the patient). Engagement device 40, along with the rest of the delivery system 11, may then be extracted from the patient.

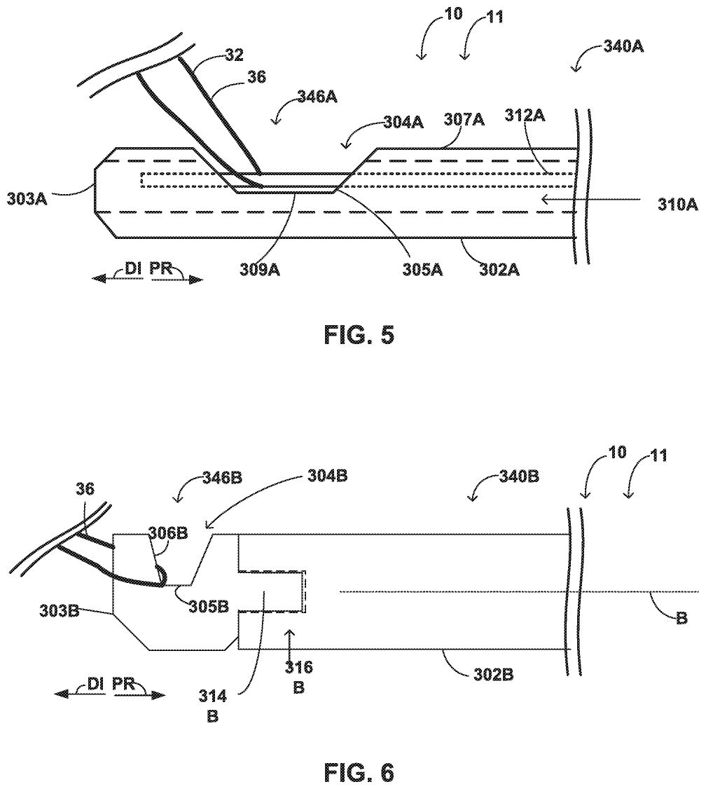

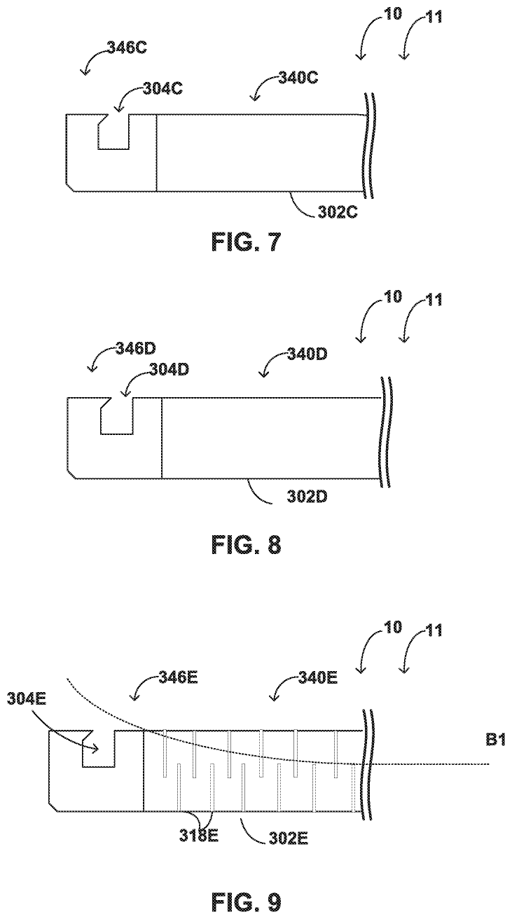

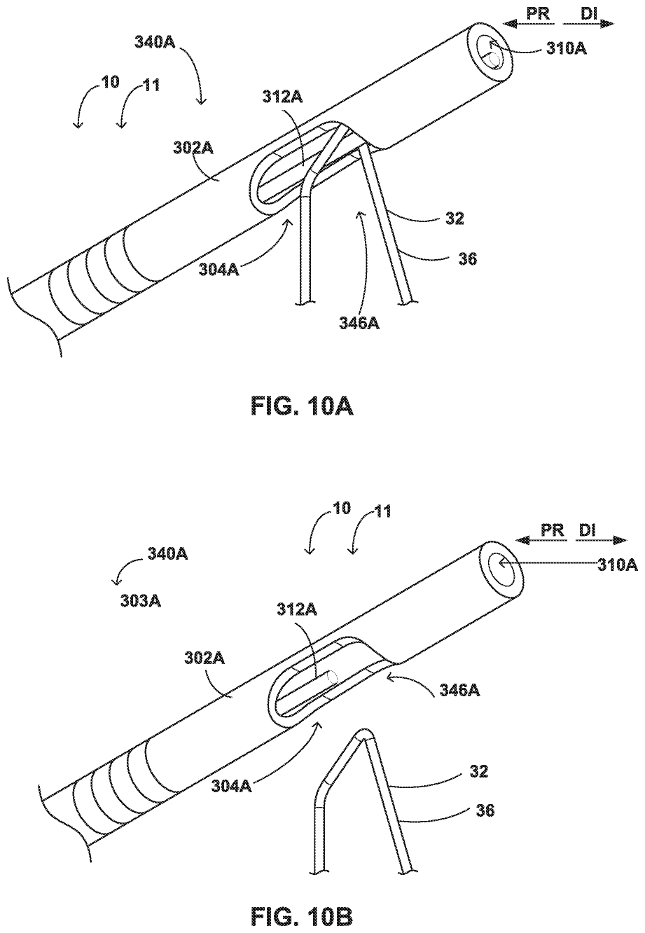

FIG. 5 is a schematic illustration of a distal portion of example engagement device 340A, taken along a central axial axis of engagement device 340A. Engagement device 340A includes release member 312A within engagement device lumen 310A defined by an engagement device body 302A. In FIG. 5, engagement device lumen 310A and portions of release member 312A within device lumen 310A are illustrated with dashed lines. FIG. 6 is schematic illustration of an example engagement device 340B, FIG. 7 is schematic illustration of an example engagement device 340C, FIG. 8 is a schematic illustration of an example engagement device 340D, and FIG. 9 is a schematic illustration of an example engagement device 340E. Engagement device 340A, engagement device 340B, engagement device 340C, engagement device 340D, and/or engagement device 340E are examples of engagement device 40 of FIGS. 1A and 1B. FIG. 10A is a perspective illustration of engagement device 340A with release member 312A in an engagement position. FIG. 10B is a perspective illustration of engagement device 340A with release member 312A in a disengagement position.

Engagement device 340A, 340B, 340C, 340D, 340E may be a portion or component of a larger delivery system, such as delivery system 11 of FIGS. 1A and 1B. In some examples, the delivery system 11 may include a delivery catheter 16 (FIGS. 1A, 1B) including a catheter body 23 (FIGS. 1A, 1B) defining a lumen such as lumen 41 (FIGS. 1A, 1B). Lumen 41 may be at least one of a main lumen 42 or an auxiliary lumen 44 defined by delivery system 11. Engagement device 340A, 340B, 340C, 340D, 340E may be configured to translate within lumen 41 to, for example, control a perimeter dimension PD defined by device 12.

Engagement device 340A, 340B, 340C, 340D, 340E may include a capture portion 346A, 346B, 346C, 346D, 346E, respectively, which may be examples of capture portion 46 (FIG. 1A, 1B). Capture portion 346A, 346B, 346C, 346D, 346E may be configured to engage with and/or disengage from cinch member 32 (e.g., second body portion 36) of device 12. Engagement device 340A, 340B, 340C, 340D, 340E may be configured such that capture portion 346A, 346B, 346C, 346D, 346E may engage with and/or engage from cinch member 32 within a heart chamber and/or other anatomical volume of a patient. Engagement device 340A, 340B, 340C, 340D, 340E may be configured such that a clinician may cause (e.g., using release mechanism 51 (FIG. 1A, 1B)) the capture portion to engage with and/or disengage from cinch member 32 when the capture portion is within the heart chamber and/or other anatomical volume of the patient. For example, a clinician may cause the capture portion to engage second body portion 36 such that engagement device 340A, 340B, 340C, 340D, 340E may transmit a force from the clinician to second body portion 36 (e.g., to control a perimeter dimension PD of device 12). A clinician may cause the capture portion to disengage from second body portion 36 to, for example, allow expansion of device 12.

For example, referring to FIGS. 5, 10A, 10B, engagement device 340A may include capture portion 346A defined by engagement device body 302. Capture portion 346A is an example of capture portion 46. Engagement device body 302 may define a recess 304A configured to receive some portion of cinch member 32 (e.g., second body portion 36) when capture portion 346A engages cinch member 32. In examples, capture portion 346A includes a release member 312A configured to engage with and/or disengage from cinch member 32. Engagement device body 302A may define a lumen 310A (“engagement device lumen 310A”) configured to allow the translation of release member 312A therethrough. In examples, engagement device body 302A includes a recess boundary 305A surrounding and/or defining at least some portion of recess 304A. In examples, engagement device body 302A defines recess 304A such that recess 304A provide a passage between an exterior surface 307A of engagement device body 302A and an interior surface 309A of engagement device body 302A. In examples, interior surface 309A at least partially defines lumen 310A.

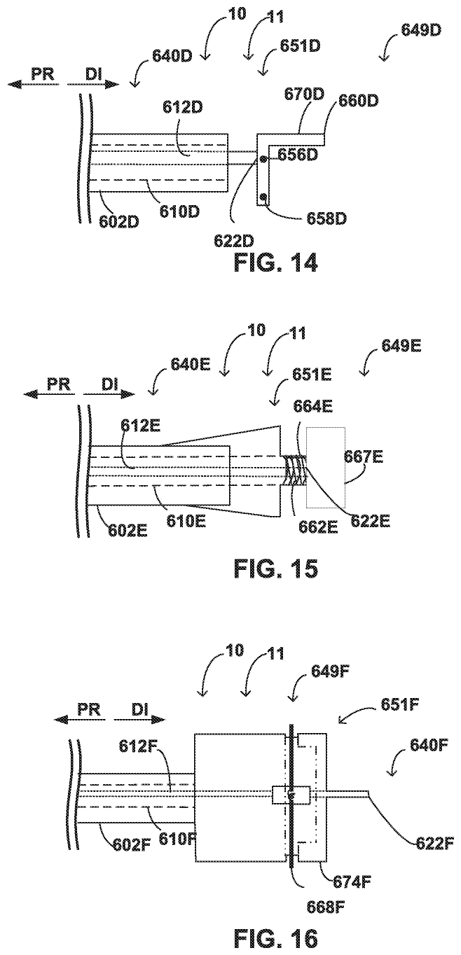

Engagement device body 302A may be a substantially elongated member configured to translate within lumen 41 of delivery system 11. Engagement device 340A (e.g., engagement device body 302A) is configured to span some portion of the length of delivery catheter 16 (FIGS. 1A and 1B). Engagement device 340A (e.g., engagement device body 302A) may be configured such that the clinician may interact with proximal portion 49 (FIGS. 1A, 1B), as discussed below with respect to FIGS. 14-19, to cause capture portion 346A to interact with second body portion 36. In some examples, engagement device body 302A may span the length of the deliver catheter (16, FIGS. 1A and 1B) as a continuous integral component. In some examples, engagement device body 302A may comprise several joined components, and may include any number of intervening components. In some examples, engagement device body 302A may be a hypotube. In some examples, engagement device body 302A may comprise stainless steel.

Recess 304A is configured to receive the second body portion 36 when capture portion 346A engages second body portion 36. Recess 304A may be, for example, a notch, groove, or other cutout portion of engagement body 302A configured to receive second body portion 336A.

Engagement device lumen 310A may open into recess 304A. In some examples, engagement device lumen 310A may open to distal end 303A. Alternatively, in some examples, engagement device lumen 310A may terminate within engagement device body 302A at a location proximal to distal end 303A. Release member 312A may be configured to translate within engagement device lumen 310A to establish an engagement position (illustrated in FIG. 5 and FIG. 10A) when release member 312A extends distal of recess 304A and/or recess boundary 305A. Release member 312A may be configured to translate within engagement device lumen 310A to establish a disengagement position (FIG. 10B) when release member 312A is retracted to a position proximal to at least some portion of recess 304A and/or recess boundary 305A. In the engagement position, release member 312A may be configured to mechanically engage cinch member 32 such that release member 312A may transfer a force exerted on engagement device body 302A to the cinch member 32. In examples, release member 312A is configured to substantially trap cinch member 32 within recess 304A when release member 312A mechanically engages cinch member 32. Hence, when a clinician exerts a force on engagement device body 302A (e.g., via proximal portion 49), release member 312A transmits the force to cinch member 32 to control perimeter dimension PD of device 12. In the disengagement position, release member 312A may be configured to mechanically disengage from cinch member 32 (e.g., by displacing from cinch member 32), such that cinch member 32 may exit recess 304A. Hence, when a clinician (e.g., using release mechanism 51) causes release member 312A to assume the disengagement position, device 12 may be free to expand (e.g., increase a perimeter dimension PD) substantially free of restraint by cinch member 32. In some examples, release member 312 includes stainless steel or nitinol, or combinations thereof.

In this way, release member 312A may be configured to engage second body portion 36 in the engagement position and disengage from the second body portion 36 in the disengagement position. Accordingly, engagement device 340A, when engaged with second body portion 36, may be translated in a distal direction to increase perimeter dimension PD established by device perimeter P, as illustrated by FIGS. 1A and 1B. Release member 312A may be retracted in a proximal direction within engagement device lumen 310A (e.g., using release mechanism 51 (FIG. 1A, 1B)) within engagement device lumen 310A to disengage device 12 from delivery system 11 (FIGS. 1A and 1B). Devices and systems of the present disclosure may accordingly afford a clinician more control of an implanted medical device through the control of the expansion of the device within a patient and reduced risk of dislodging the device upon successful deployment through the use of engagement device 340A.

FIG. 6 is a schematic illustration of example engagement device 340B. Engagement device 340B may be similar to engagement device 340A, where like numbers indicate like elements.

Engagement device 340B includes capture portion 346B defining recess 304B. Recess 304B may be a notch, groove, or other cutout configured to receive second body portion 36. In examples, engagement device body 302A includes a recess surface 305B substantially surrounding and/or defining at least some portion of recess 304B. Recess surface 305B may include a bearing surface 306B. Bearing surface 306B may be configured to transmit a force from an engagement device body 302A of engagement device 340B to second body portion 36 when engagement device 340B exerts a force on the second body portion 36. In examples, recess surface 305B and/or bearing surface 306B define a hook-like structure, such that recess 304B substantially defines a throat of the hook.

For example, a clinician may cause a pulling force to be exerted in the proximal direction on engagement device 340B. Bearing surface 306B may transmit the force to second body portion 36, causing second body portion to cinch first body portion (34, FIGS. 2-3B) to cinch device 12 (FIGS. 1A-4). To disengage engagement device 340B from device 12, the clinician may cause engagement device body 302B to rotate about axis B defined by engagement device body 302B and/or otherwise cause recess 304B to displace from second body portion 36, causing second body portion 36 to exit recess 304B of capture portion 346B and thus freeing (e.g., disengaging) device 12 from delivery system 11 (e.g., engagement device 340B). In this way, a hook-like structure may comprise the capture portion 46 of some example engagement devices 40 of a delivery system 11.

Engagement device 340B (e.g., recess surface 305B and/or bearing surface 306B) may define recess 304B such that recess 304B may substantially capture second body portion 36. For example, engagement device 340B may be configured such that a rotation of engagement device 304 B about axis B and/or a displacement of recess 304B toward second body portion 36 (e.g., by a clinician) causes recess 304B to receive second body portion 36. Bearing surface 306B may engage (e.g., contact) second body portion 36 when recess 304B receives second body portion, such that engagement device 340B may exert a force on second body portion 36 (e.g., via bearing surface 306B).

In examples, capture portion 346B is a separately machined section of engagement device 340B joined to (e.g., welded, crimped-on, or otherwise joined) to engagement device body 302B. In some examples, engagement device body 302B defines one of an aperture (e.g., aperture 316B) or a protrusion and capture portion 346B defines the other of the aperture or the protrusion (e.g., protrusion 314B). Engagement device body 303B and capture portion 346B may be configured such that the aperture receives the protrusion when capture portion 346B is joined with engagement device body 302B. In other examples, capture portion 346B and engagement device body 303B may be portions of a unified body comprising engagement device 340B.

FIG. 7 illustrates an engagement device 340C including an engagement device body 302C. FIG. 8 illustrates an engagement device 340D including an engagement device body 302D. FIG. 9 illustrates an engagement device 340E including an engagement device body 302E. Engagement device body 302C, engagement device body 302D, and engagement device body 302E are examples of engagement device body 302A, 302B. Engagement device 340C, 340D, 340E may be similar to engagement device 340A, 340B, where like numbers indicate like elements.

Engagement device body 302A, 302B, 302C, 302D, 302E may be configured to flex and/or bend (e.g., define a curved or curvilinear shape) in order to cause capture portion 46, 346A, 346B, 346C, 346D, 346E to engage with, disengage from, or otherwise exert a force on cinch member 32. Engagement device body 302A, 302B, 302C, 302D, 302E may be configured to flex from configuration defining axis B (FIG. 6) to a configuration defining a more curved axis such as axis B1 (FIG. 9). For example, as illustrated in FIG. 7, engagement device body 302C may include a coil (e.g., a helical coil) configured to flex and/or bend. In some examples, as illustrated in engagement device 340D of FIG. 8, engagement device body 302D may include a polymer material, such as a medical-grade polymer. The polymer may, for example, include a polyimide, polyether ether ketone (PEEK), combinations thereof, or the like. In some examples, as illustrated in FIG. 9, engagement device 340E includes engagement device body 302E, which may include a series of cutouts or slots 318E configured to reduce the resistance of engagement device body 302E to flexing and/or bending. In this way or other ways, engagement device body 302A, 302B, 302C, 302D, 302E may be configured to flex (e.g., define a curved or curvilinear shape) in order to cause capture portion 46, 346A, 346B, 346C, 346D, 346E to engage with and/or disengage from cinch member 32.

In examples, delivery catheter proximal portion 15, engagement device proximal portion 49, and/or another portion of delivery system 11 is configured to cause engagement device body 302A, 302B, 302C, 302D, 302E to flex. For example, delivery catheter proximal portion 15, engagement device proximal portion 49, and/or another portion of delivery system 11 may include a steering system configured to be operated by a clinician to cause engagement device body 302A, 302B, 302C, 302D, 302E to flex.

In some examples, second body portion 36 may be inserted into recess 304A, 304B, 304C, 304D, 304E as part of a packing or pre-loading process. For example, second body portion 36 may be inserted into recess 304A, 304B, 304C, 304D, 304E during loading of device 12 in delivery capsule 18 (FIG. 1A, 1B). In some examples, delivery capsule 18 may be configured to restrain device 12 in the delivery configuration. Second body portion 36 may be inserted into recess 304A, 304B, 304C, 304D, 304E such that engagement device 340A, 340B, 340C, 340D, 340E substantially traps and/or otherwise constrains second body portion 36 within recess 304A, 304B, 304C, 304D, 304E. For example, as part of a packing or pre-loading process, second body portion 36 may be looped around release member 312A, and release member 312A may be slidably translated within lumen 310A in a distal direction beyond recess 304A, establishing an engagement position which locks second body portion 36 into recess 304A. Hence, medical system 10 may be configured such that engagement device 340A, 340B, 340C, 340D, 340E is engaged with cinch member 32 as device 12 is deployed from delivery capsule 18, easing burden on a clinician during a procedure.

In some examples, a control unit (not pictured) may control or actuate a translation of engagement device 40 within lumen 41 in either a proximal or distal direction. Similarly, a control unit may control or actuate a translation in either a proximal or distal direction of the release member 312A within an engagement device lumen 310A defined by engagement device 40. Additionally or alternatively, a clinician may control or actuate a translation of engagement device 40 within lumen 41 and/or release member 312A within engagement device lumen 310A manually. Any suitable electrical, mechanical, or electromechanical system may assist a clinician in starting, stopping, and/or managing the translation of engagement device 40 and/or release member 312A within a lumen of medical system 100 (e.g., lumen 41 and/or engagement device lumen 310A).

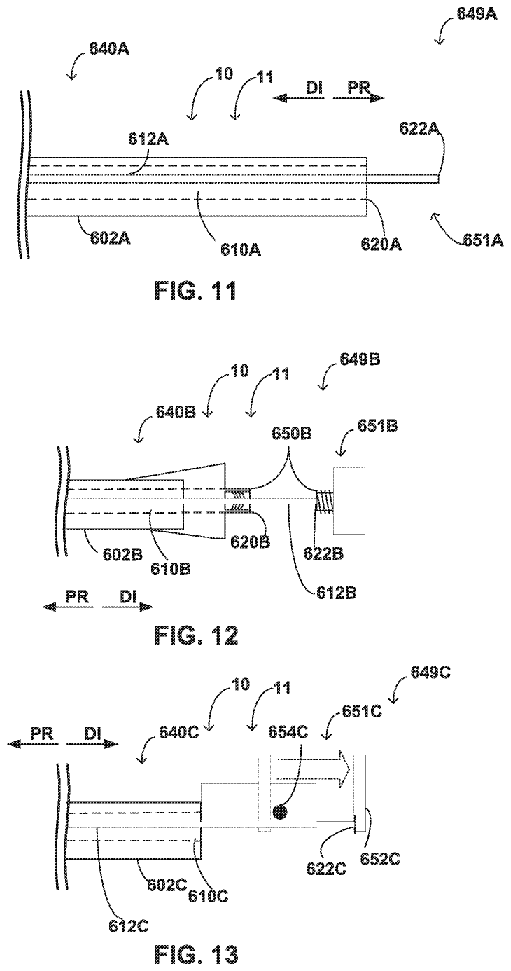

FIGS. 11-16 are schematic side views illustrating example engagement device proximal portions according to the present disclosure, illustrating a number of non-limiting options to mechanically assist a clinician in starting, stopping, and/or managing the translation of an example release member. Combinations of the following examples and alternatives to the following examples are considered. In FIGS. 11-16, engagement device lumen 610A, 610B, 610C, 610D, 610E, 610F and portions of release member 612A, 612B, 612C, 612D, 612E, 612F within engagement device lumen 610A, 610B, 610C, 610D, 610F are illustrated with dashed lines.

FIG. 11 is schematic illustration of an example engagement device 640A including an engagement device proximal portion 649A and a release member 612A within engagement device lumen 610A. FIG. 12 is a schematic illustration of a proximal portion 649B of an example engagement device 640B, including a release mechanism 651B coupled to a release member 612B within a lumen 610B. FIG. 13 is a schematic illustration of a proximal portion 649C of an example engagement device 640C, including a release mechanism 651C coupled to a release member 612C within a lumen 610C. FIG. 14 is a schematic illustration of a proximal portion 649D of an example engagement device 640D, including a release mechanism 651D coupled to a release member 612D within a lumen 610D. FIG. 15 is a schematic illustration of a proximal portion 649E of an example engagement device 640E, including a release mechanism 651D coupled to a release member 612D within a lumen 610D. FIG. 16 is a schematic illustration of a proximal portion 649F of an example engagement device 640F, including a release mechanism 651F coupled to a release member 612B within a lumen 610B. Engagement device 640A, 640B, 640C, 640D, 640E, 640F are examples of engagement device 40, 340A, 340B, 340C, 340D, 340E, 340F (FIGS. 1A, 1B). Engagement device proximal portion 649A, 649B, 649C, 649D, 649E, 649F are examples of engagement device proximal portion 49 (FIGS. 1A, 1B). Release member 612A, 612B, 612C, 612D, 612E, 612F are examples of release member 312A (FIGS. 5, 10A, 10B). Engagement device lumen 610A, 610B, 610C, 610D, 610E, 610F are examples of engagement device lumen 310A (FIGS. 5, 10A, 10B). Release mechanism 651A, 651B, 651C, 651D, 651E, 651F are examples of release mechanism 51 (FIGS. 1A, 1B).

In examples, engagement device 640A, 640B, 640C, 640D, 640E, 640F is a portion or component of a larger delivery system, such as delivery system 11 of FIGS. 1A and 1B. In some examples, the delivery system 11 may include a delivery catheter 16 (FIGS. 1A, 1B) including a catheter body 23 (FIGS. 1A, 1B) defining a lumen such as lumen 41. (FIGS. 1A, 1B). Lumen 41 may be at least one of a main lumen 42 or an auxiliary lumen 44 defined by delivery system 11. Engagement device 640A, 640B, 640C, 640D, 640E, 640F may be configured to translate within lumen 41 to, for example, control a perimeter dimension PD defined by device 12. Engagement device 640A, 640B, 640C, 640D, 640E. 640F may be configured such that a clinician may cause (e.g., using release mechanism 51 (FIG. 1A, 1B)) capture portion 46, 346A, 346B, 346C, 346D, 346E to engage with and/or disengage from cinch member 32 when capture portion 46, 346A, 346B, 346C, 346D, 346E is within the heart chamber and/or other anatomical volume of the patient. For example, a clinician may interact with engagement device proximal portion 649A, 649B, 649C, 649D, 649E, 649F to cause the capture portion 46, 346A, 346B, 346C, 346D, 346E to engage second body portion 36 such that engagement device 40, 640A, 640B, 640C, 640D, 640E, 640F may transmit a force from the clinician to second body portion 36 (e.g., to control a perimeter dimension PD of device 12). A clinician may cause the capture portion to disengage from second body portion 36 to, for example, allow expansion of device 12.

FIG. 11 illustrates proximal portion 649A of example engagement device 640A. Engagement device 640A includes a capture portion (346A, FIGS. 5, 10A, 10B). Capture portion 346A may engage second body portion (36, FIGS. 5, 10A, 10B) of cinch member 32 (346A, FIGS. 5, 10A, 10B) as described above with respect to FIGS. 5 and 10A and B. Release member 612A may be configured to translate in a proximal direction (illustrated by arrow PR) or distal direction (illustrated by arrow DI) within engagement device lumen 610A. In some examples, release member 612A may extend through a proximal end 620A (e.g., through an opening of engagement device lumen 610A) of engagement device body 602A. Engagement device 640A may be configured such that movement of proximal end 622A of release member 612A (e.g., by a clinician) in a distal direction causes engagement device 640A to establish the engagement position (FIG. 10A) where second body portion 36 is captured by release member 612A. Engagement device 640A may be configured such that movement of proximal end 622A of release member 612A in a proximal direction (e.g., by a clinician) in a proximal direction causes engagement device 640A to establish the disengagement position (FIG. 10B) in which second body portion 36 is released or freed from engagement device 640A. Accordingly, in some examples release mechanism 651A may be proximal end 622A of release member 612A which extends beyond proximal end 620A of engagement device body 602A and is configured to translate within engagement device lumen 610A.

FIG. 12 illustrates engagement device proximal portion 649B. Release mechanism 651B may include a threaded connection 650B. Threaded connection 650B is provided between proximal end 620B of engagement device body 602B and proximal end 622B of release member 612B. A threaded connection may be desirable to prevent release member 612B from establishing the disengagement position prior to the clinician being ready to release a device (e.g., device 12) from the delivery system (e.g., delivery system 11. Threaded connection 650B may reduce the risk of the release mechanism (e.g., release mechanism 51, FIGS. 1A and 1B) from being activated accidentally during a medical procedure.

Threaded connection 650B is configured such that release member 612B may threadably engage engagement device proximal portion 649B (e.g., when engagement device 640 is in the engagement position). Threaded connection 650B may be configured such that, when release member 612B is threadably engaged with engagement device proximal portion 649B, rotation of release member 612B in a first rotational direction causes translation of release member 612B in a distal direction relative to engagement device proximal portion 649B. Threaded connection 650B may be configured such that, when release member 612B is threadably engaged with engagement device proximal portion 649B, rotation of release member 612B in a second rotational direction opposite the first rotational direction causes translation of release member 612B in a proximal direction relative to engagement device proximal portion 649B. In examples, proximal end 620B defines one of a threaded protrusion or a threaded recess and proximal end 622B defines the other of the threaded protrusion or the threaded recess. The threaded recess may be configured to receive the threaded protrusion when proximal end when release member 612B is threadably engaged with engagement device proximal portion 649B.

FIG. 13 is a close up view of a proximal portion 649C of an example engagement device 640C. Release mechanism 651C may include a release lever 652C and/or safety pin 654C. In this example, release lever 652C may be connected at proximal 622C of release member 612C. Release lever 652C may be configured to be engaged and or disengaged from a safety pin 654C, which may be mounted on a proximal portion of a delivery device, handle, or other supporting structure. Release lever 652C may be locked in the distal position (illustrated in broken lines) by safety pin 654C, which may correspond to the engagement position of the capture portion (FIG. 10A). In some examples, locking release lever 652C in the distal position (and thus release member 612C in the engagement position) may include engaging safety pin 654C with release lever 652C.

In some examples, engaging release lever 652C with safety pin 654C may prevent release member 612C from translating to the disengagement position (FIG. 10B), and thus prevent capture portion (not pictured) of engagement device 640C from releasing a second body portion (e.g., second body portion 36) of a cinch member (e.g., cinch member 32) of a device (e.g., device 12) prematurely, because a clinician or supporting staff member must actively disengage safety pin 654C before release member 612C may be translated in a proximal direction to establish the disengaged position (FIG. 10B).

FIG. 14 is a close up view of a proximal portion 649D of example engagement device 640D. Release mechanism 651D may include a release member 612D and/or push-release component 660D. In the illustrated example, a push-release mechanism for actuating translation of release member 612D is provided. Push-release component 660D may be coupled to proximal end 622D of release member 612D, and in some examples push-release component 660D may be configured to rotate about stationary pivot 658D upon actuation by a clinician. In some examples, actuation by a clinician may involve application of a force on surface 670D of push-release component 660D. Rotation of push-release component 660D about stationary pivot 658D may cause movable pivot 656D to move along a travel (not pictured) and accordingly cause release member 612D to move proximally from the engagement position (FIG. 10A) to the disengagement position (FIG. 10B). In some examples, inclusion of a push release mechanism as illustrated in FIG. 14 may mechanically assist a clinician in controlling the position of release member 612D.

FIG. 15 is a close up view of a proximal portion 649E of example engagement device 640E. Release mechanism 651E may include a spring mechanism 662E. Spring mechanism 662E may include a spring 664E and a spring block 667E. In some examples, spring 664E may be compressed and locked in place using a safety pin mechanism (FIG. 13), threaded connection (FIG. 14), or other suitable release mechanism. In some examples, release of the release mechanism may cause spring 664E to urge against spring block 667 (e.g., expand from a compressed condition). Spring block 667 may be coupled to proximal end 622E of release member 612E, such that the expansion of spring 664E from the compressed condition causes translation (e.g., proximal translation) of spring block 667 and release member 612E. Accordingly, a spring mechanism 662E may assist a clinician in releasing second body portion 36 from engagement device 40 in some examples by applying a spring force to release member 612E in the proximal direction.

FIG. 16 is a close up view of a proximal portion 649F of example engagement device 640F. Release mechanism 651F may include a pin block 674F. FIG. 16 illustrates an example push-pin mechanism for preventing accidental release of a device (e.g., device 12) from engagement device 640F including pin block 674F. In some examples, pin block 674F may be configured to hold release member 612F in place until a clinician or supporting staff member pushes on or more pins 668F into a recess (illustrated in broken lines) within pin block 674F to allow release member 612F to translate proximally from the engagement position (FIG. 10A) to the disengagement position FIG. 10B.

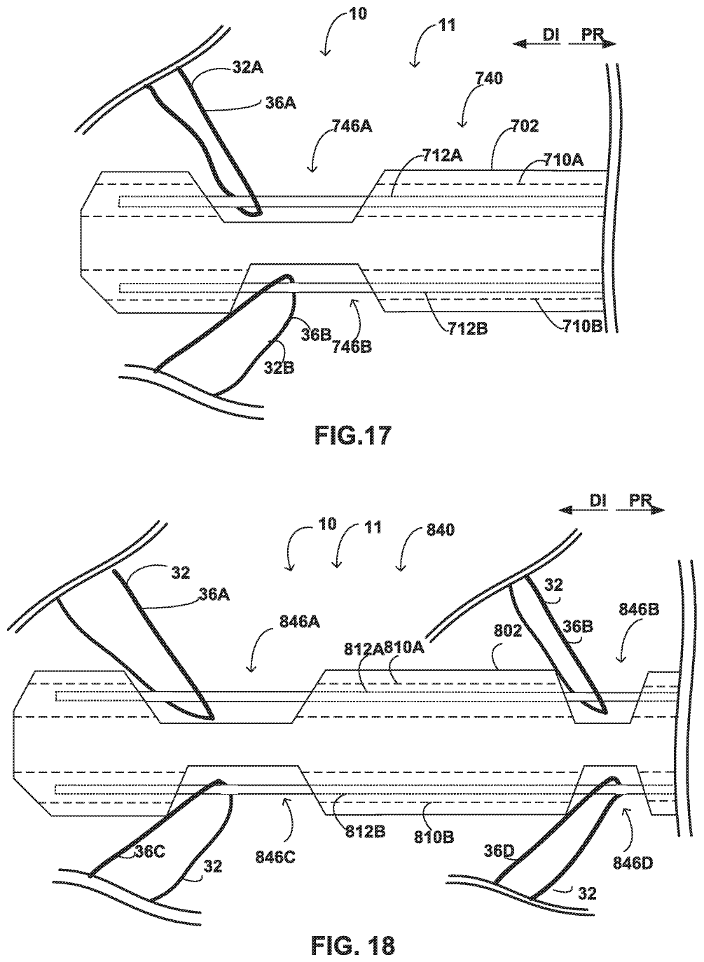

In some examples, as illustrated by FIG. 17 a device (e.g., device 12) may include a plurality of cinch members (e.g., cinch members 32A and 32B). FIG. 17 illustrates an example engagement device 740 from a schematic side view. In FIG. 17, engagement device lumens 710A, 710B and portions of release member 712A, 712B within engagement device lumens 710A, 710B are illustrated with dashed lines. Engagement device 740 is an example of engagement device 40 illustrated in FIGS. 1A and 1B.

In some examples, each cinch member 32A, 32B may include a corresponding first body portion (not pictured) and second body portion 36A, 36B. Additionally or alternatively, a cinch member (e.g., cinch member 32) may include a plurality of first body portions and second body portions 36A, 36B. In any case, a device including more than one second body portion 36A, 36B is considered, whether the more than one second body portions 36A, 36B are defined by a single cinch member (e.g., cinch member 32, FIGS. 1A and 1B) or multiple cinch members 32A, 32B. Accordingly, a medical system 10 may include a delivery system 11 which may include an engagement device 740 configured to engage more than one second body portion 36A, 36B of a cinch member 32A, 32B. In some examples, engagement device 740 may include capture portion 746A and capture portion 746B, although any number of capture portions may be included (e.g., two, three, or four capture portions). Inclusion of multiple capture portions 746A, 746B and multiple corresponding second body portions 36A, 36B may provide a clinician additional control over deployment and/or retraction of a device (e.g., device 12) relative to a delivery system (e.g. delivery system 11) which includes only one capture portion (e.g., capture portion 46) and only one second body portion (e.g., second body portion 36). In some examples, multiple capture portions 746A, 746B may be disposed about a circumference of an engagement device body 702, which may provide for a more centered device (e.g., device 12). In some examples, a more centered device may be desirable to reduce the risk of mispositioning or dislodgment of the device during delivery or deployment.

Referring to FIG. 18, in some examples a medical system 10 may include a delivery system 11 which may include engagement device 840. Engagement device 840 is an example of engagement device 40 illustrated in FIGS. 1A and 1B. Engagement device 840 may define any number of capture portions 846A, 846B, 846C, 846D, and at least two of the capture portions are spaced apart along an axial direction of engagement device 840. For example, as illustrated in FIG. 18, capture portion 846A and capture portion 846B are spaced apart along an axial axis of engagement device 840 such that release member 812A may translate within engagement device lumen 810A to establish an engaged position and a disengaged position of capture portion 846A, and an engaged position and a disengaged position of capture portion 846B.

In some examples, each capture portion 846A, 846B, 846C, 846D may be configured to engage with a second body portion 36A, 36B, 36C, 36D of a cinch member 32. In some examples, engagement device 840 may be configured to disengage individual capture portions 846A, 846B, 846C, 846D progressive succession as release member 812 translates in a proximal direction PR. In some examples, inclusion of capture portions 846A, 846B spaced apart along an axial direction of engagement device 840 may allow a clinician more control over deployment and/or retraction of device 12.

With reference to FIGS. 1A and 1B, additional examples are described. In some examples, a delivery system 11 may include a plurality of engagement devices 40, and each of the plurality of engagement devices 40 may be configured to engage a corresponding second body portion 36 of a cinch member 32. In some examples, each of the plurality of engagement devices (e.g., engagement device 40) may be configured to translate within a corresponding auxiliary lumen (e.g. auxiliary lumen 44) defined by the delivery catheter 16, and the plurality of auxiliary lumens (e.g., auxiliary lumen 44) may be disposed about main lumen 42 in different radial directions. Accordingly, more control over the perimeter dimension (PD, FIG. 2) of a device perimeter (P, FIG. 2) may be gained, yielding a more successfully deployment and/or retraction of a device (e.g., device 12), and reduced risk of dislodgment. Furthermore, a delivery system (e.g., delivery system 11) including more than one engagement device (e.g., engagement device 40) may allow for a more centered device expansion, because the perimeter dimension PD may be controlled by a plurality of engagement devices (e.g., engagement device 40) about the circumference of a delivery catheter 16.

In examples, as depicted in FIG. 4, outer support 39 includes a plurality of outer support struts 66, such as outer support struts 68 and 70. Outer support struts 68, 70 may be substantially elongated members configured to substantially define a shape of valve outer support 39 (e.g., in the expanded configuration and/or delivery configuration). In examples, outer support struts 68, 70 are configured to resiliently bias device 12 (e.g., outer support 39) from the delivery configuration to the expanded configuration when radially unconstrained (e.g., when cinch member 32 is at least partially relaxed and/or when delivery capsule 18 (FIGS. 1A-1B) is at least partially displaced from device 12). Cinch member 32 may be at least partially relaxed by, for example, translating engagement device 40 in a distal direction such that first body portion 34 allows radial expansion of device 12. Outer support struts 66 may be resiliently biased to expand radially outward from valve axis L. In examples, outer support struts 66 are configured to urge device 12 from the delivery configuration to the expanded configuration to cause device 12 to substantially fit into the annulus of a native heart valve. In examples, outer support struts 66 are elongated members configured to substantially define a shape of outer support 39 in the expanded configuration and/or delivery configuration. In some examples, outer support struts 68, 70 may be joined to define one or more cells such as cell 72. For example, outer support struts 68, 70 may be joined to define one or more acute angles and/or obtuse angles defining cell 72. In examples, cell 72 is a substantially diamond-shaped cell. Valve support struts 76, 78 may be configured to mechanically support other portions of device 12, such as valve assembly 43, outer support sealing member 54, and/or other portions of device 12.

Valve support 38 may include a plurality of valve support struts 74, such as valve support strut 76 and valve support strut 78. Valve support struts 76, 78 may be substantially elongated members configured to substantially define a shape of valve support 38 (e.g., in the expanded configuration and/or delivery configuration). In some examples, valve support struts 76, 78 may be joined to define one or more cells such as cell 80. For example, valve support struts 76, 78 may be joined to define one or more acute angles and/or obtuse angles defining cell 80. In examples, cell 80 is a substantially diamond-shaped cell. Valve support struts 76, 78 may be configured to mechanically support other portions of device 12, such as valve assembly 43, outer support sealing member 54, and/or other portions of device 12.

Device 12 may include valve brim 180. Valve brim 180 may include brim structures 186 such as brim structure 182 and brim structure 184. Brim structures 186 may be relatively rigid portions or members of brim 180 configured to define the shape of brim 180. FIG. 4 illustrates a portion of valve brim 180 for clarity, however valve brim 180 and brim structures 186 may substantially surround valve axis L. Valve brim 180 may be disposed at inflow region 144 of device 12 and may be a portion of outer support 39. In some examples, valve brim 180 may be mechanically supported by anchoring member 56. In some examples, valve brim 180 may configured to assist a clinician in positioning a clinician in locating and positioning device 12 during a percutaneous medical operation by increasing the visibility of prosthetic device on an imaging system (e.g., an ultrasound imaging system). In some examples, valve brim 180 extends at least partially radially outward from valve axis L relative to anchoring member 56.

In some examples, valve brim 180 may be covered by brim sealing member 57. Brim sealing member 57 may be configured to limit (e.g., substantially reduce) blood flow in a radial direction (e.g., substantially perpendicular to valve axis L) through valve brim 180. In examples, brim sealing member may comprise a flexible material, such as Dacron® or another type of polymeric material. Brim sealing member 57 may substantially cover (e.g., be in contact with) an interior surface of valve brim 180 facing valve axis L and/or an exterior surface of valve brim 180 facing away from valve axis L. In some examples, valve support sealing member 52 is attached to valve assembly 43. In some examples, brim sealing member 57 defines one or more passages 116 between brim structures 182 and 184 covered by brim sealing member 57.

In examples, device 12 is configured such that valve support 38 and/or outer support 39 surround valve axis L at when device 12 is in the expanded configuration. In examples, valve support 38 includes a substantially tubular member (e.g., tubular or nearly tubular to the extent permitted by manufacturing tolerances). Valve support 38 (e.g., valve support struts 76, 78) may define one or more upstream crowns 82 at a first end 84 of valve support 38 (“first valve support end 84”). Valve support 38 (e.g., valve support struts 76, 78) may define one or more downstream crowns 86 defining a second end 88 of valve support 38 (“second valve support end 88”) opposite first valve support end 84. Outer support 39 (e.g., anchoring member struts 68, 70) may define one or more upper crowns 90 defining a first end 92 of outer support 39 (“first anchoring end 92”). Outer support 39 (e.g., anchoring member struts 68, 70) may include one or more lower crowns 94 defining a second end 96 of outer support 39 (“second anchoring end 96”) opposite first anchoring end 92. Outer support 39 may include and/or mechanically support anchoring member 56. Anchoring member 56 may include a first portion 102 (“first fixation portion 102”) and a second portion 104 (“second fixation portion 104’). Anchoring member 56 may be configured such that first fixation portion 102 is substantially between first anchoring end 92 and second fixation portion 104. Anchoring member 56 may be configured such that second fixation portion 104 is substantially between second anchoring end 96 and first fixation portion 102. In examples, anchoring member 56 (e.g., first fixation portion 102 and/or second fixation portion 104) defines a substantially annular shape surrounding valve axis L. In examples, anchoring member 56 defines a substantially annular shape surrounding valve support 38 and/or valve assembly 43.

As discussed, member body 33 defines a closed loop configured to control (e.g., reduce) a perimeter dimension PD defined by a device perimeter P (e.g., perimeter P1 and/or perimeter P2) defined by device 12. Perimeter P1, P2 may be defined by any portion of device 12. For example, perimeter P1 may be defined by brim 180, and/or one or more of brim structures 186. Perimeter P2 may be defined by one or more first anchoring ends such as first anchoring end 92, one or more upper crowns such as upper crown 90, one or more anchor support struts such as anchoring member struts 68, 70, one or more second anchoring ends such as second anchoring end 96, and/or one or more lower crowns such as lower crown 94. In some examples, perimeter P1, P2 may be defined by one or more first valve support ends such as first valve support end 84, one or more upstream crowns such as upstream crown 82, one or more valve support struts such as valve supports struts 76, 78, one or more second valve support ends such as second valve support end 88, and/or one or more downstream crowns such as downstream crown 86.

In examples, outer support 39 includes a base 106 attached to outflow region 146 of valve support 38. In some examples, second anchoring end 96, second valve support end 88, downstream crowns 86, and/or lower crowns 94 are joined with and/or define base 106. In examples, the plurality of anchoring member struts (e.g., anchoring member struts 68, 70) define a plurality of arms 108 projecting radially outward (relative to valve axis L) from base 106. Anchoring member 56 may extend from arms 108. In examples, anchoring member 56 is configured such that, when device 12 is in the expanded configuration (as shown, for example, in FIGS. 1B, 2, 3A, 3B), anchoring member 56 and/or outer support 39 is spaced radially outward apart from valve support 38 by the gap G. When prosthetic heart valve is in a delivery configuration (FIG. 1A), gap G may be reduced or substantially eliminated. In examples, anchoring member 56 includes a ring (e.g., a cylindrical or conical ring). Anchoring member 56 may define an engagement surface 110 configured to press outwardly against the native annulus. In examples, engagement surface 110 mechanically supports a plurality of anchoring elements 58 projecting radially outward from engagement surface 110. In examples, one or more of anchoring elements 58 may be inclined toward an upstream direction (e.g., inclined in a direction from outflow region 146 to inflow region 144). The anchoring elements 58, for example, can be barbs, hooks, cleats, tines, or other elements configured to engage tissue when device 12 is in the expanded configuration within heart 14 (FIG. 1A).

Valve support sealing member 52 is configured to limit (e.g., substantially reduce) blood flow in a radial direction (e.g., substantially perpendicular to valve axis L) through valve support 38. Outer support sealing member 54 is configured to limit blood flow in a radial direction (e.g., substantially perpendicular to valve axis L) through outer support 39. In examples, valve support sealing member 52 and/or outer support sealing member 54 may be made from a flexible material, such as Dacron® or another type of polymeric material. Valve support sealing member 52 may substantially cover (e.g., be in contact with) an interior surface of valve support 38 facing valve axis L and/or an exterior surface of valve support 38 facing away from valve axis L. Outer support sealing member 54 may substantially cover (e.g., be in contact with) an interior surface of outer support 39 facing valve axis L and/or an exterior surface of outer support 39 facing away from valve axis L. In some examples, valve support sealing member 52 is attached to valve assembly 43. In examples, outer support sealing member 54 is attached to outer support 39 such that anchoring elements 58 are uncovered by outer support sealing member 54, such that, for example, anchoring elements 58 may engage tissue when device 12 is in the expanded configuration. In some examples, valve support sealing member 52 and/or outer support sealing member 54 may include more than one layer, such as two layers. In some examples, one or more passages 116 may be configured to be part of valve support sealing member 52 or outer support sealing member 54, such as by a sewn or otherwise defined passage through two layers of sealing member ((e.g., valve support sealing member 52)).

Device 12 may be configured to replace any native heart valve. In examples, device 12 may be configured to replace a previously implanted prosthetic heart valve.

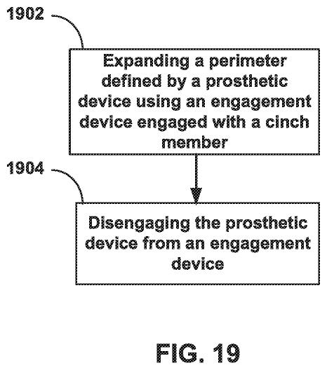

FIG. 19 is a flow chart illustrating an example technique for delivering a prosthetic device. Although the technique is described primarily with reference to medical system 10 including delivery system 11 and device 12 of FIGS. 1A-18, the technique may be applied to other prosthetic devices in other examples.

The technique includes expanding a device perimeter P defined by device 12 (1902). In some examples, device 12 includes an outer support 39 configured to engage an annulus of a heart valve of a heart 14. Outer support 39 may cause a valve axis L defined by device 12 to pass through the annulus when the outer support 39 engages the annulus. Device 12 includes a valve support 38 mechanically supported by outer support 39 and surrounding the valve axis L. Valve support 38 may define a flow path from an inflow region 144 of the valve support 38 to an outflow region 146 of the valve support 38. Device 12 further includes valve assembly 43 mechanically supported by valve support 38 within the flow path. Valve assembly 43 may allow a blood flow through the flow path and valve assembly 43 when device 12 is positioned within an annulus of a heart valve.

A delivery capsule 18 may substantially enclose device 12 to limit the expansion of device 12 (e.g., expansion in a direction away from valve axis L). The technique may include displacing a capsule housing 28 from device 12 (e.g., when delivery capsule is in proximity to an annulus of a heart valve) to allow at least a partial expansion of device 12. Cinch member 32 may control the expansion of device 12 (e.g., control a perimeter dimension of a perimeter P1, P2 defined by device 12). In examples, engagement device 40 exerts a force on second body portion 36 to cause cinch member 32 to control the expansion of device 12. Engagement device 40 may engage cinch member 32 using capture portion 46 to exert the force.

In examples, expanding a perimeter P defined by device 12 comprises translating first body portion 34 within passage 116 (e.g., using engagement device 40 engaged with second body portion 36). In some examples, expanding a perimeter P defined by device 12 comprises expanding device perimeter P in a direction substantially perpendicular to valve axis L. In some examples, expanding a perimeter P defined by device 12 includes reducing a displacement between engagement device 40 and some portion of device 12 (e.g., outer support 39. In some examples, expanding device perimeter P includes translating engagement device 40 towards first body portion 34. In some examples, outer support 39 includes anchoring member 56 and valve brim 180. Anchoring member 56 may engage the annulus of a heart valve. Anchoring member 56 may mechanically support valve brim 180. In examples, one of the valve brim 180, the anchoring member 56, or the valve support 38 defines the device perimeter P. In some examples the one of the valve brim 180, the anchoring member 56, or the valve support 38 defining the device perimeter P also defines and/or supports the one or more passages 116.

The technique may include disengaging device 12 from engagement device 40 (1904). Device 12 may disengage cinch member 32 from capture portion 46 to disengage device 12 from engagement device 40. In examples, capture portion 46 receives second body portion 36 within recess 304A when capture portion 46 engages second body portion 36. Second body portion 36 may exit recess 304A when capture portion 46 disengages from second body portion 36. In examples, release member 312A substantially traps second body portion 36 in recess 304A when capture portion 46 engages second body portion 36. Release member 312A may displace from second body portion 36 (e.g., translate relative to recess 304A) to cause capture portion 46 to disengage from second body portion 36. In some examples, engagement device 40 includes a bearing surface (306B, FIG. 6) defining the recess (304B, FIG. 6). In some examples, the bearing surface may be configured to transmit a force from engagement device 40 when engagement device 40 exerts the force on second body portion 36.

Disengaging device 12 from capture portion 46 of engagement device 40 may include slidably translating release member (412, FIG. 10A) from an engagement position (FIG. 10A) to a disengagement position (FIG. 10B). In some examples, engagement device 40 defines an engagement device lumen (310A, FIG. 5) opening into the recess (304A, FIG. 5). Release member 312A may be configured to engage second body portion 36 in the engagement position (FIG. 10A) and disengage from the second body portion in the disengagement position (FIG. 10B).

In some examples, disengaging device 12 from capture portion 46 of engagement device 12 may further include actuating and/or operating a release mechanism 51 disposed at proximal portion 49 of engagement device 40. Release mechanism 51 may include one or more of release mechanism 651A, 651B, 651C, 651D, 651E, and/or 651F as illustrated by FIGS. 11-16. In some examples, actuating a release mechanism (651A, FIG. 11) may include manually causing a proximal end (622A, FIG. 11) of a release member (612A, FIG. 11) to translate in a proximal direction.

In some examples, actuating a release mechanism (651B, FIG. 12) may include engaging a threaded connection (650B, FIG. 12), which may be disposed between a proximal end (620B, FIG. 12) of an engagement device body (602B, FIG. 12) and a proximal end (622B, FIG. 12) of the release member (612B, FIG. 12). In some examples, rotation of the release member (612B, FIG. 12) in a first rotational direction may cause translation of release member (612B, FIG. 12) in a distal direction relative to the engagement device proximal portion (649B, FIG. 12). Rotation of the release member (612B, FIG. 12) in a second rotational direction opposite the first rotational direction may cause translation of the release member (612B, FIG. 12) in a proximal direction relative to the engagement device proximal portion (649B, FIG. 12).

In some examples, actuating the release mechanism (651C, FIG. 13) may include engaging and/or disengaging a release lever (652C, FIG. 13) from a safety pin (654C, FIG. 13). In some examples, actuating the release mechanism (651D, FIG. 14) may include applying a force on a surface (670D, FIG. 14) of a push-release component (660D, FIG. 14). In some examples, application of a force on the surface (670D, FIG. 14) may cause the release member (612D, FIG. 14) to translate from the engagement position (FIG. 10A) to the disengagement position (FIG. 10B).

In some examples, actuating the release member (651E, FIG. 15) may include causing a spring (662E, FIG. 15) to urge against a spring block (664E, FIG. 15). In some examples, the spring (664E, FIG. 15) may be compressed and locked in place using a safety pin mechanism (FIG. 13), threaded connection (FIG. 14), or other suitable release mechanism. In some examples, actuating the release mechanism (651F, FIG. 16) may include actuating a push pin mechanism which may include one or more pins (668F, FIG. 16) within a pin block (674F, FIG. 16).

Example 1: A system includes a prosthetic device to engage an annulus of a heart valve of a heart, wherein the prosthetic device is configured to cause a valve axis defined by the prosthetic device to pass through the annulus when the prosthetic device engages the annulus; and a cinch member including a member body including a first body portion and a second body portion defining a closed loop, wherein the first body portion is configured to translate within one or more passages defined by the prosthetic device, wherein the prosthetic device defines a device perimeter surrounding the valve axis, and wherein the first body portion is configured to reduce a perimeter dimension of the device perimeter when an engagement device exerts a force on the second body portion.

Example 2, The system of example 1, wherein the prosthetic device includes an outer support configured to engage the annulus, and wherein the outer support is configured to cause the valve axis to pass through the annulus when the outer support engages the annulus.

Example 3: The system of example 1 or example 2, wherein the prosthetic device includes a valve support surrounding the valve axis, wherein the valve support is configured to define a flow path from an inflow region of the valve support to an outflow region of the valve support.

Example 4: The system of example 3, wherein the valve support is mechanically supported by the outer support of example 2.

Example 5: The system of any of example 3 or example 4, wherein the prosthetic device includes a valve assembly mechanically supported by the valve support within the flow path, wherein the valve assembly is configured to allow a blood flow through the flow path.

Example 6: The system of any of examples 1-5, wherein the cinch member is configured to cause the first body portion to slidably translate within the one or more passages to reduce the perimeter dimension when the engagement device exerts the force on the second body portion.

Example 7: The system of any of examples 1-6, wherein the perimeter dimension is measured in a direction substantially perpendicular to the valve axis.

Example 8: The system of example 2, wherein the cinch member is configured to displace the outer support toward the valve axis when the engagement device exerts the force on the second body portion.

Example 10: The system of example 9, wherein the first body portion is configured to overcome the resilient biasing when the engagement device exerts the force on the second body portion.

Example 11: The system of any of examples 1-10, wherein the cinch member is configured to reduce the perimeter dimension when the engagement device exerts the force on the second body portion in a direction away from the first body portion.

Example 12: The system of example 2, wherein the outer support includes an anchoring member configured to engage the annulus and a brim mechanically supported by the anchoring member, wherein one of the brim, the anchoring member, or the valve support defines the device perimeter.

Example 13: The system of example 12, wherein the one of the brim, the anchoring member, or the valve support define the one or more passages.

Example 14: The system of any of examples 1-13, wherein the prosthetic device is configured to establish a first perimeter dimension in a delivery configuration and a second perimeter dimension in an expanded configuration, wherein the second perimeter dimension is greater than the first perimeter dimension.

Example 15: The system of any of examples 1-14, further comprising the engagement device, wherein the engagement device includes an engagement device body defining a capture portion, wherein the capture portion is configured to engage the second body portion and is configured to disengage from the second body portion.

Example 16: The system of example 15, wherein the capture portion defines a recess configured to receive the second body portion when the capture portion engages the second body portion.

Example 17: The system of example 16, wherein the engagement device body includes a bearing surface defining the recess, and wherein the bearing surface is configured to transmit a force from the engagement device body when the engagement device exerts the force on the second body portion.

Example 18: The system of example 16, wherein the engagement device body defines an engagement device lumen opening into the recess, the engagement device body further comprising a release member configured to slidably translate within the lumen to establish an engagement position and a disengagement position, wherein the release member is configured to engage the second body portion in the engagement position and disengage from the second body portion in the disengagement position.

Example 19: The system of any of examples 15-18, further comprising a delivery catheter including a catheter body defining a lumen, and wherein the engagement device is configured to translate within the lumen.

Example 20: The system of example 19, wherein the delivery catheter includes a delivery capsule configured to restrain the prosthetic heart valve in the delivery configuration of example 10.

Example 21: The system of example 20, wherein delivery catheter includes a deployment member supporting the delivery capsule, wherein the deployment member is configured to translate within the lumen to displace the at least some portion of the delivery capsule from the prosthetic device.

Example 22: The system of any of examples 19-21, wherein the lumen is at least one of a main lumen or an auxiliary lumen, wherein the catheter body defines the main lumen and the auxiliary lumen, and wherein the deployment member of example 17 is configured to translate within the main lumen and the engagement device of any of examples 11-17 is configured to translate within the auxiliary lumen.

Example 23: A system for delivering a heart valve prosthesis includes an engagement device including an engagement device body defining a capture portion; and a prosthetic device configured to engage an annulus of a heart valve of a heart, wherein the prosthetic device is configured to cause a valve axis defined by the prosthetic device to pass through the annulus when the prosthetic device engages the annulus; a cinch member including a member body including a first body portion and a second body portion defining a closed loop, wherein the first body portion is configured to translate within one or more passages defined by the prosthetic device, wherein the prosthetic device defines a device perimeter surrounding the valve axis, wherein the first body portion is configured to reduce a perimeter dimension of the device perimeter when the capture portion engages the second body portion and the engagement device exerts a force on the second body portion, and wherein the capture portion is configured to disengage from the second body portion.

Example 24: The system of example 23, wherein the prosthetic device includes an outer support configured to engage the annulus, and wherein the outer support is configured to cause the valve axis to pass through the annulus when the outer support engages the annulus.

Example 25: The system of example 23 or example 24, wherein the prosthetic device includes a valve support surrounding the valve axis, wherein the valve support is configured to define a flow path from an inflow region of the valve support to an outflow region of the valve support.

Example 26: The system of example 25, wherein the valve support is mechanically supported by the outer support of example 24.

Example 27: The system of any of example 25 or example 26, wherein the prosthetic device includes a valve assembly mechanically supported by the valve support within the flow path, wherein the valve assembly is configured to allow a blood flow through the flow path.

Example 28: The system of any of examples 23-27, wherein the capture portion defines a recess configured to receive the second body portion when the capture portion engages the second body portion.

Example 29: The system of example 28, wherein engagement device body includes a bearing surface defining the recess, and wherein the bearing surface is configured to transmit a force from the engagement device body when the engagement device exerts the force on the second body portion.

Example 30: The system of example 28, wherein engagement device body defines an engagement device lumen opening into the recess, and further comprising a release member configured to slidably translate within the lumen to establish an engagement position and a disengagement position, wherein the release member is configured to engage the second body portion in the engagement position and disengage from the second body portion in the disengagement position.

Example 31: The system of any of examples 23-30, further comprising a delivery system including a catheter body defining a lumen, and wherein the engagement device is configured to translate within the lumen.

Example 32: The system of example 31, wherein the delivery catheter includes a delivery capsule configured to restrain the prosthetic heart valve in a delivery configuration.

Example 33: The system of example 32, wherein delivery catheter includes a deployment member supporting the delivery capsule, wherein the deployment member is configured to translate within the lumen to displace the delivery capsule from the prosthetic device.

Example 34: The system of any of examples 31-33, wherein the lumen is at least one of a main lumen or an auxiliary lumen, wherein the catheter body defines the main lumen and the auxiliary lumen, and wherein the deployment member of example 25 is configured to translate within the main lumen and the engagement device of any of examples 19-25 is configured to translate within the auxiliary lumen.

Example 35: A method of delivering a prosthetic device includes expanding a perimeter defined by prosthetic device using an engagement device engaged with a cinch member, the prosthetic device configured to engage an annulus of a heart valve of a heart, wherein the prosthetic device is configured to cause a valve axis defined by the prosthetic device to pass through the annulus when the prosthetic device engages the annulus; and the cinch member, wherein the cinch member includes a member body including a first body portion and a second body portion defining a closed loop, wherein the first body portion is disposed within one or more passages defined by the prosthetic device, wherein the second body portion is configured to be engaged by an engagement device, wherein the prosthetic device defines a device perimeter surrounding the valve axis, and wherein the first body portion is configured to reduce a perimeter dimension defined by the device perimeter when the engagement device exerts a force on the second body portion; and disengaging the prosthetic device from the engagement device.

Example 36: The method of example 35, wherein expanding the perimeter comprises translating the first body portion within the one or more passages.

Example 37: The method of example 35 or example 36, wherein expanding the perimeter comprises expanding the perimeter in a direction substantially perpendicular to the valve axis.

Example 38: The method of any of examples 35-37, wherein expanding the perimeter comprises translating the engagement device towards the first body portion.

Example 39: The method of any of examples 35-38, wherein expanding the perimeter comprises displacing an outer support from the valve axis, wherein the outer support is defined by the prosthetic device and configured to cause the valve axis to pass through the annulus when the prosthetic device engages the annulus.

Example 40: The method of example 39, wherein displacing the outer support from the valve axis comprises translating the engagement device within a lumen defined by the delivery catheter.

Example 41: The method of example 40, wherein reducing the force on the second body portion comprises moving the engagement device towards the second body portion.

Example 42: The method of any of examples 39-41, wherein the outer support includes an anchoring member configured to engage the annulus and a brim mechanically supported by the anchoring member, wherein one of the brim, the anchoring member, or the valve support defines the device perimeter.

Example 43: The method of example 42, wherein the one of the brim, the anchoring member, or the valve support define the one or more passages.

Example 44: The method of any of examples 35-43, wherein expanding the perimeter comprises increasing the perimeter dimension from a first perimeter dimension to a second perimeter dimension, wherein the second perimeter dimension is greater than the first perimeter dimension.

Example 45: The method of any of examples 35-44, wherein disengaging the prosthetic device from the engagement device comprises disengaging the prosthetic device from a capture portion of an engagement device body, wherein the capture portion is configured to engage the second body portion and configured to disengage from the second body portion.

Example 46: The method of example 45, further comprising positioning the second body portion within a recess defined by the capture portion when the capture portion engages the second body portion.

Example 47: The method of example 46, wherein the engagement device body includes a bearing surface defining the recess, and further comprising transmitting a force from the engagement device body to the second body portion when the engagement device exerts the force on the second body portion.

Example 48: The method any of examples 35-47, wherein disengaging the prosthetic device from a capture portion of an engagement device body comprises slidably translating a release member from an engagement position to a disengagement position, wherein the release member is configured to engage the second body portion in the engagement position and disengage from the second body portion in the disengagement position.

Example 49: The method of any of examples 35-48, further comprising a delivery system including a delivery catheter defining a lumen, wherein the lumen is at least one of a main lumen or an auxiliary lumen, and wherein the engagement device is configured to translate within the lumen.

Example 50: The method of example 49, further comprising displacing a delivery capsule from the prosthetic device.

Example 51: The method of example 50, wherein displacing the delivery capsule comprises translating a deployment member of the delivery catheter, wherein the deployment member supports the delivery capsule, and wherein the deployment member is configured to translate within the lumen to displace the delivery capsule from the prosthetic device.

Example 52: The method of any of examples 49-51, wherein a catheter body defines the main lumen and the auxiliary lumen, and wherein the deployment member of example 42 is configured to translate within the main lumen and the engagement device is configured to translate within the auxiliary lumen.

Example 53: The method of any of examples 35-52, further comprising reducing the perimeter, wherein reducing the perimeter comprises reducing the perimeter dimension from an initial perimeter dimension to a subsequent perimeter dimension, wherein the subsequent perimeter dimension is less than the initial perimeter dimension.

Various examples of the disclosure have been described. Any combination of the described systems, operations, or functions is contemplated. These and other examples are within the scope of the following claims.

1. A system comprising:
a prosthetic device configured to engage an annulus of a heart valve of a heart, wherein the prosthetic device is configured to cause a valve axis defined by the prosthetic device to pass through the annulus when the prosthetic device engages the annulus; and
a cinch member including a member body including a first body portion and a second body portion defining a closed loop,
wherein the first body portion is permanently attached to the prosthetic device and is configured to translate within one or more passages defined by the prosthetic device,
wherein the prosthetic device defines a device perimeter surrounding the valve axis, and
wherein the first body portion is configured to reduce a perimeter dimension of the device perimeter when an engagement device exerts a force on the second body portion.

2. The system of claim 1, wherein the prosthetic device includes an outer support configured to engage the annulus, wherein the outer support is configured to cause the valve axis to pass through the annulus when the outer support engages the annulus, and wherein the prosthetic device includes a valve support surrounding the valve axis, wherein the valve support is configured to define a flow path from an inflow region of the valve support to an outflow region of the valve support.

3. (canceled)

4. (canceled)

5. The system of claim 2, wherein the prosthetic device includes a valve assembly mechanically supported by the valve support within the flow path, wherein the valve assembly is configured to allow a blood flow through the flow path.

6. The system of claim 1, wherein the cinch member is configured to cause the first body portion to slidably translate within the one or more passages to reduce the perimeter dimension when the engagement device exerts the force on the second body portion.

7. The system of claim 1, wherein the perimeter dimension is measured in a direction substantially perpendicular to the valve axis.

8. The system of claim 2, wherein the cinch member is configured to displace the outer support toward the valve axis when the engagement device exerts the force on the second body portion.

9. The system of 8, wherein the prosthetic device is resiliently biased to resist the displacement of the outer support toward the valve axis.

10. (canceled)

11. The system of claim 1, wherein the cinch member is configured to reduce the perimeter dimension when the engagement device exerts the force on the second body portion in a direction away from the first body portion.

12. The system of claim 2, wherein the outer support includes an anchoring member configured to engage the annulus and a brim mechanically supported by the anchoring member, wherein one of the brim, the anchoring member, or the valve support defines the device perimeter, and wherein the one of the brim, the anchoring member, or the valve support define the one or more passages.

13. (canceled)

14. The system of claim 1, wherein the prosthetic device is configured to establish a first perimeter dimension in a delivery configuration and a second perimeter dimension in an expanded configuration, wherein the second perimeter dimension is greater than the first perimeter dimension.

15. The system of claim 1, further comprising the engagement device, wherein the engagement device includes an engagement device body defining a capture portion, wherein the capture portion is configured to engage the second body portion and is configured to disengage from the second body portion.

16. The system of claim 15, wherein the capture portion defines a recess configured to receive the second body portion when the capture portion engages the second body portion.

17. The system of claim 16, wherein the engagement device body includes a bearing surface defining the recess, and wherein the bearing surface is configured to transmit a force from the engagement device body when the engagement device exerts the force on the second body portion.

18. The system of claim 16, wherein the engagement device body defines an engagement device lumen opening into the recess, the engagement device body further comprising a release member configured to slidably translate within the lumen to establish an engagement position and a disengagement position, wherein the release member is configured to engage the second body portion in the engagement position and disengage from the second body portion in the disengagement position.

19. The system of claim 15, further comprising a delivery catheter including a catheter body defining a lumen, and wherein the engagement device is configured to translate within the lumen.

20. The system of claim 19, wherein the delivery catheter includes a delivery capsule configured to restrain the prosthetic heart valve in the delivery configuration.

21. The system of claim 20, wherein delivery catheter includes a deployment member supporting the delivery capsule, wherein the deployment member is configured to translate within the lumen to displace at least some portion of the delivery capsule from the prosthetic device.

22. The system of claim 21, wherein the lumen is at least one of a main lumen or an auxiliary lumen, wherein the catheter body defines the main lumen and the auxiliary lumen, and wherein the deployment member is configured to translate within the main lumen and the engagement device is configured to translate within the auxiliary lumen.

23. A system for delivering a heart valve prosthesis, the system comprising:
an engagement device including an engagement device body defining a capture portion; and
a prosthetic device configured to engage an annulus of a heart valve of a heart, wherein the prosthetic device is configured to cause a valve axis defined by the prosthetic device to pass through the annulus when the prosthetic device engages the annulus,
wherein the prosthetic device includes a cinch member including a member body defining a first body portion and a second body portion,
wherein the first body portion and the second body portion define a closed loop,
wherein the first body portion is configured to translate within one or more passages defined by the prosthetic device,
wherein the prosthetic device defines a device perimeter surrounding the valve axis,
wherein the first body portion is configured to reduce a perimeter dimension of the device perimeter when the capture portion engages the second body portion and the engagement device exerts a force on the second body portion, and
wherein the capture portion is configured to disengage from the second body portion.

24. A method of delivering a prosthetic device, the method comprising:
delivering the prosthetic device to a heart valve of a heart with the prosthetic device in a delivery configuration with a perimeter having a first perimeter dimension;
expanding the perimeter to a second perimeter dimension larger than the first perimeter dimension using an engagement device engaged with a cinch member such that the prosthetic device engages an annulus of the heart valve of the heart and a valve axis defined by the prosthetic device passes through the annulus when the prosthetic device engages the annulus; and
disengaging the cinch member from the engagement device such that the prosthetic device disengages from the engagement device and the cinch member remains with the prosthetic device.
